(19) United States
(12) Patent Application Publication
(10) Pub. No.: US 2025/0346580 A1
(43) Pub. Date: Nov. 13, 2025

(54) NOVEL PYRIMIDINYL SULFONAMIDE DERIVATIVES

(71) Applicant: Hoffmann-La Roche Inc., Little Falls, NJ (US)

(72) Inventors: Guido GALLEY, Rheinfelden (DE); Luca GOBBI, Buus (CH); Dmitry MAZUNIN, Grenzach-Wyhlen (DE); Emmanuel PINARD, Linsdorf (FR)

(73) Assignee: Hoffmann-La Roche Inc., Little Falls, NJ (US)

(21) Appl. No.: 19/272,823

(22) Filed: Jul. 17, 2025

Related U.S. Application Data
(63) Continuation of application No. PCT/EP2024/051094, filed on Jan. 18, 2024.

(30) Foreign Application Priority Data
Jan. 20, 2023 (EP) 23152565.0

(51) Int. Cl. C07D 403/14; A61K 31/506; C07B 59/00; C07D 401/14; C07D 403/12; C07D 417/14

(52) U.S. Cl. CPC C07D 403/14; A61K 31/506; C07B 59/002; C07D 401/14; C07D 403/12; C07D 417/14

(57) ABSTRACT

The invention relates to novel compounds having the general formula I

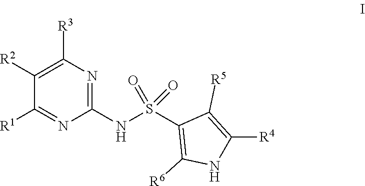

wherein R¹, R², R³, R⁴, R⁵ and R⁶ are as described herein, composition including the compounds and methods of using the compounds.

### CROSS-REFERENCE TO RELATED APPLICATIONS

This application is a Continuation of International Application No. PCT/EP2024/051094, filed Jan. 18, 2024, which claims benefit of priority to European Application No. 23152565.0 filed Jan. 20, 2023, each of which is incorporated herein by reference in its entirety.

The present invention relates to organic compounds useful for therapy and/or prophylaxis in a mammal, and in particular to compounds that modulate GPR17 activity.

The present invention provides novel compounds of formula I

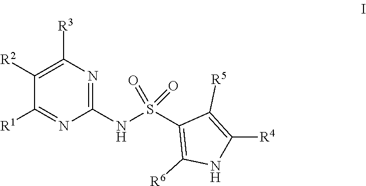

wherein,
R¹ is alkoxy or haloalkoxy;
R² is halo, alkyl, alkoxy, haloalkyl, haloalkoxy, cyanoalkyl, cyanoalkoxy, or cyclopropyl optionally substituted with up to two substituents independently selected from cyano and halo;
R³ is H, alkoxy, or haloalkoxy;
R⁴ is H, cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with oxo, halo, alkyl, haloalkyl, alkoxy, haloalkoxy, or cyano;
R⁵ is H, halo, alkyl, aryl, heteroaryl, arylalkyl or heteroarylalkyl wherein aryl, heteroaryl, arylalkyl or heteroarylalkyl are optionally substituted with halo, alkyl, haloalkyl, alkoxy, haloalkoxy, or cyano;
R⁶ is H or halo;
and pharmaceutically acceptable salts.

Furthermore, the invention includes all racemic mixtures, all their corresponding enantiomers and/or optical isomers.

### BACKGROUND OF THE INVENTION

Myelination is a process that occurs robustly during development and despite the abundant presence of oligodendrocyte precursor cells (OPCs) throughout the adult CNS, the transition to myelinating oligodendrocytes and the production of restorative myelin sheaths around denuded axons is impaired in chronic demyelinating diseases. During development, myelination proceeds in a very orderly manner, with OPCs, characterized by expression of markers such as neural/glial antigen 2 (NG2) and platelet-derived growth factor alpha (PDGFRα), differentiating into oligodendrocytes which lose NG2 and PDGFRα expression and gain the expression of markers such as myelin basic protein (MBP) and myelin oligodendrocyte glycoprotein (MOG). The production of myelin by oligodendrocytes is a very tightly regulated process and in the CNS, this can be controlled by interactions with axons, well-understood in the peripheral but not in the central nervous system (Macklin, W. B. (2010). Sci. Signal. 3, pe32-pe32, “The myelin brake: When Enough Is Enough”). Myelination can also be controlled by internal brakes within oligodendrocytes themselves, through the transcription factor EB (TFEB)-PUMA axis or through GPR17 antagonism (Chen, Y., et al. (2009). Nat Neurosci 12, 1398-1406, “The oligodendrocyte-specific G protein-coupled receptor GPR17 is a cell-intrinsic timer of myelination”) (Sun, L. O., et al. (2018). Cell 175, 1811-1826.e21, “Spatiotemporal Control of CNS Myelination by Oligodendrocyte Programmed Cell Death through the TFEB-PUMA Axis”). Myelin serves not only to protect axons and facilitate neuronal transmission, but oligodendrocytes have also been shown to play an important role in metabolism of axons as well as in maintaining the electrolyte balance around axons (Schirmer, L., et al. (2014). Ann Neurol 75, 810-828, “Differential loss of KIR4.1 immunoreactivity in multiple sclerosis lesions”) (Simons, M., and Nave, K.-A. (2015). Cold Spring Harb Perspect Biol. 22, “Oligodendrocytes: Myelination and Axonal Support”).

GPR17 is a Class A orphan G protein-coupled receptor (GPCR). GPCRs are 7 domain transmembrane proteins that couple extracellular ligands with intracellular signaling via their intracellular association with small, heterotrimeric G-protein complexes consisting of G_{α}, G_{β}, G_{Υ} subunits. It is the coupling of the GPCR to the G_{α} subunit that confers results in downstream intracellular signaling pathways. GPR17 is known to be coupled directly to G_{α i/o}, which leads to inhibition of adenylate cyclase activity, resulting in a reduction in cyclic AMP production (cAMP). GPR17 has also been shown to couple to G_{q/11}, that targets phospholipase C. Activation of phospholipase C leads to the cleavage of phosphatidylinositol 4,5-bisphosphate which produces inositol triphosphate (IP₃) and diacylglycerol (DAG). IP₃ consequently binds to the IP₃ receptor on the endoplasmic reticulum and causes an increase in intracellular calcium levels (Hanlon, C. D., and Andrew, D. J. (2015). J Cell Sci. 128, 3533-3542, “Outside-in signaling—a brief review of GPCR signaling with a focus on the Drosophila GPCR family”) (Inoue, A., et al. (2019), Cell 177, 1933-1947.e25, “Illuminating G-Protein-Coupling Selectivity of GPCRs”).

The role of GPR17 in myelination was first identified in a screen of the optic nerves of Olig1 knockout mice to identify genes regulating myelination. GPR17 expression was found to be expressed only in the myelinating cells of the CNS and absent from the Schwann cells, the peripheral nervous system's myelinating cells. The expression of GPR17 was found to be exclusively expressed in the oligodendrocyte lineage cells and was downregulated in myelinating oligodendrocyte (Chen, Y., et al. (2009)). Specifically, GPR17 expression is found to be present at low levels early on in the OPC and increases in the pre-myelinating oligodendrocyte before the expression is downregulated in the mature, myelinating oligodendrocyte (Boda, E., et al. (2011), Glia 59, 1958-1973, “The GPR17 receptor in NG2 expressing cells: Focus on in vivocell maturation and participation in acute trauma and chronic damage”) (Dziedzic, A., et al. (2020). Int. J. Mol. Sci. 21, 1852, “The gpr17 receptor—a promising goal for therapy and a potential marker of the neurodegenerative process in multiple sclerosis”) (Fumagalli, M. et al. (2011), J Biol Chem 286, 10593-10604, “Phenotypic changes, signaling pathway, and functional correlates of GPR17-expressing neural precursor cells during oligodendrocyte differentiation”). GPR17 knockout animals were shown to exhibit precocious myelination throughout the CNS and conversely, transgenic mice overexpressing GPR17 in oligodendrocytes with the CNP-Cre (2′, 3′-cyclic-nucleotide 3′-phosphodiesterase) promoter exhibited myelinogenesis defects, in line with what is to be expected of a cell-intrinsic brake on the myelination process (Chen, Y., et al. (2009)). Furthermore, loss of GPR17 enhances remyelination following demyelination with lysophosphatidylcholine-induced demyelination (Lu, C., Dong, et al. (2018), Sci. Rep. 8, 4502, “G-Protein-Coupled Receptor Gpr17 Regulates Oligodendrocyte Differentiation in Response to Lysolecithin-Induced Demyelination”). As such, antagonism of GPR17 that promotes the differentiation of oligodendrocyte lineage cells into mature, myelinating oligodendrocytes would lead to increase in myelination following demyelination.

Multiple sclerosis (MS) is a chronic neurodegenerative disease that is characterized by the loss of myelin, the protective fatty lipid layer surrounding axons, in the central nervous system (CNS). Prevention of myelin loss or remyelination of denuded axons is thought to prevent axonal degeneration and thus prevent progression of the disease (Franklin, R. J. (2002), Nat Rev Neurosci 3, 705-714, “Why does remyelination fail in multiple sclerosis?”). Due to the restorative impact that myelin repair has on the central nervous system, such a treatment will benefit all types of MS namely relapse-remitting, secondary progressive, primary progressive and progressive relapsing MS. Reparation of lost myelin will alleviate neurological symptoms associated with MS due to the neuroprotective effect of preserving axons.

Due to the essential role that myelination plays in functioning of the nervous system, facilitating OPC to oligodendrocyte differentiation has the potential to impact multiple diseases where white matter defects/irregularities due to either loss of myelinating oligodendrocytes or hampered differentiation of OPCs to oligodendrocytes have been observed, due to the disease itself or inflammation. This is in addition to the diseases where GPR17 expression itself is altered.

The diseases that GPR17 antagonism can be thus used to yield a positive disease outcome include, but are not limited to:

### Direct Damage to Myelin Sheaths:

Metabolic conditions that lead to destruction of central myelin such as central pontine myelinolysis, extra-pontine myelinolysis due to overly-rapid correction of hyponatremia in conditions for instance, but not limited to, alcoholism, liver disease, immunosuppression after transplantation
Carbon monoxide poisoning where oligodendrocyte dysfunction and failure to regenerate has been reported in the deep white matter layers of the brain
Nutritional deficiency that results in myelin loss or failure to properly generate myelin during development
Virus-induced demyelination

### Primary Demyelinating Disorders

Multiple Sclerosis (relapse-remitting, secondary progressive, primary progressive and progressive relapsing MS)
Acute and multiphasic disseminated encephalomyelitis
Neuromyelitis optica spectrum disorders including optic neuritis
Transverse myelitis
Leukodystrophies such as adrenoleukodystrophy, adrenomyeloneuropathy and other inherited leukodystrophies that result in myelin loss
   CNS Disorders with Associated Myelin Loss:
Alzheimer's Disease
Schizophrenia
Parkinson's Disease
Huntington's disease
Amyotrophic lateral
Ischemia due to stroke

### Other Diseases:

Inflammation in the CNS for instance following encephalitis, primary angiitis, meningitis

The compounds of formula I bind to and modulates GPR17 activity.

The compounds of formula I are therefore particularly useful in the treatment of diseases related to GPR17 antagonism.

The compounds of formula I are particularly useful in the treatment or prophylaxis of multiple sclerosis (MS), conditions related to direct damage to myelin sheaths such as carbon monoxide poisoning or virus induced demyelination, primary demyelinating disorders such as acute and multiphasic disseminated encephalomyelitis, and other CNS disorders associated with myelin loss such as Alzheimer's disease, schizophrenia, Parkinson's disease and Huntington's disease.

### SUMMARY OF THE INVENTION

The present invention provides novel compounds of formula I

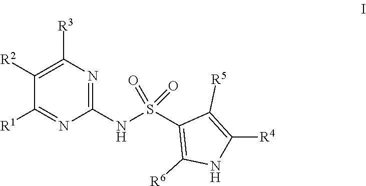

wherein,
R¹ is alkoxy or haloalkoxy;
R² is halo, alkyl, alkoxy, haloalkyl, haloalkoxy, cyanoalkyl, cyanoalkoxy, or cyclopropyl optionally substituted with up to two substituents independently selected from cyano and halo;
R³ is H, alkoxy, or haloalkoxy;
R⁴ is H, cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with oxo, halo, alkyl, haloalkyl, alkoxy, haloalkoxy, or cyano;
R⁵ is H, halo, alkyl, aryl, heteroaryl, arylalkyl or heteroarylalkyl wherein aryl, heteroaryl, arylalkyl or heteroarylalkyl are optionally substituted with halo, alkyl, haloalkyl, alkoxy, haloalkoxy, or cyano;
R⁶ is H or halo;
and pharmaceutically acceptable salts.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl group is methyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular example is methoxy.

The term “halogen”, “halide” and “halo” are used interchangeably herein and denote fluoro, chloro, bromo or iodo. Examples of “halo” include fluoro.

The term “haloalkyl” denotes a C₁₋₆-alkyl group wherein at least one of the hydrogen atoms of the C₁₋₆-alkyl group has been replaced by the same or different halogen atoms. Particular example is difluoroethyl.

The term “haloalkoxy” denotes a C₁₋₆-alkoxy group wherein at least one of the hydrogen atoms of the C₁₋₆-alkoxy group has been replaced by the same or different halogen atoms. Particular examples fluoroethoxy, difluoroethoxy, and difluoromethoxy.

The term “cyano” denotes a —C≡N group.

“Cyanoalkyl” means a moiety of the formula —R′—R″, where R′ is alkyl as defined herein and R″ is cyano or nitrile. Particular example is cyanoethyl.

“Cyanoalkoxy” means a moiety of the formula —R′—R″, where R′ is alkoxy as defined herein and R″ is cyano or nitrile.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Particular examples of a cycloalkyl are cyclopropyl and cyclobutyl.

The term “heterocycle ring” or “heterocycle” denotes a monovalent saturated or partly unsaturated mono- or bicycle ring system of 4 to 10 ring atoms, or 4 to 9 ring atoms, comprising 1, 2, or 3 ring heteroatoms selected from N, O and S, the remaining ring atoms being carbon.

The term “aryl”, alone or in combination with other groups, denotes a monovalent cyclic aromatic hydrocarbon moiety consisting of a mono- or bicyclic aromatic ring. Preferred aryl is phenyl. Aryl may be unsubstituted or substituted as described herein.

The term “arylalkyl” denotes an alkyl group wherein one of the hydrogen atoms of the alkyl group has been replaced by an aryl group. Examples of arylalkyl are benzyl.

The term “heteroaryl” denotes a monovalent aromatic mono- or bicyclic ring system of 4 to 9 ring atoms, comprising 1, 2, 3, or 4 ring heteroatoms selected from N and O, the remaining ring atoms being carbon. Bicyclic means consisting of two cycles having one or two ring atoms in common. Example for heteroaryl are thiazolyl and pyridyl. Other examples of heteroaryl are pyrimidinyl, pyridazinyl, and pyrazinyl

The term “heteroarylalkyl”, denotes an alkyl group wherein one of the hydrogen atoms of the alkyl group has been replaced by a heteroaryl group.

The term “oxo” denotes a divalent oxygen atom ═O.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is alkoxy.

An embodiment of the present invention provides compounds according to formula I as described herein, wherein R² is wherein R² is haloalkyl, haloalkoxy, cyanoalkyl or cyclopropyl substituted with cyano.

An embodiment of the present invention provides compounds according to formula I as described herein, wherein R² is haloalkyl or haloalkoxy.

An embodiment of the present invention provides compounds according to formula I as described herein, wherein R³ is H or alkoxy.

An embodiment of the present invention provides compounds according to formula I as described herein, wherein R³ is alkoxy.

An embodiment of the present invention provides compounds according to formula I as described herein, wherein R⁴ is selected from cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with, halo, alkyl, haloalkyl, alkoxy, haloalkoxy, or cyano.

An embodiment of the present invention provides compounds according to formula I as described herein, wherein R⁴ is a 6-membered-aryl, a 5- or 6-membered heteroaryl comprising 1-to-2 heteroatoms independently selected from S and N, arylalkyl or heteroarylalkyl, wherein a 6-membered-aryl, a 5- or 6-membered heteroaryl comprising 1-to-2 heteroatoms independently selected from S and N, arylalkyl or heteroarylalkyl are optionally substituted with halo, alkyl, haloalkyl, alkoxy, haloalkoxy, or cyano.

An embodiment of the present invention provides compounds according to formula I as described herein, wherein R⁴ is selected from
i. H;
ii. Cyclobutyl;
iii. 6-membered aryl;
iv. A 5-membered heteroaryl comprising 1 S heteroatom and 1 N heteroatom;
v. A 6-membered heteroaryl comprising 1 or 2 N heteroatoms;
vi. A 6-member aryl substituted with halo;
vii. A 5-membered heteroaryl comprising 1 S heteroatom and 1 N heteroatom substituted by alkyl;
viii. A 6-membered heteroaryl comprising 1 N heteroatom substituted with alkyl, halo, or both alkyl and oxy.

An embodiment of the present invention provides compounds according to formula I as described herein, wherein R⁴ is selected from
i. 6-membered aryl;
ii. A 5-membered heteroaryl comprising 1 S heteroatom and 1 N heteroatom;
iii. A 6-membered heteroaryl comprising 1 N heteroatoms;
iv. A 6-member aryl substituted with halo;
v. A 5-membered heteroaryl comprising 1 S heteroatom and 1 N heteroatom substituted by alkyl;
vi. A 6-membered heteroaryl comprising 1 N heteroatom substituted with alkyl or halo.

An embodiment of the present invention provides compounds according to formula I as described herein, wherein R⁴ is a 6-membered-aryl, or a 5-or-6-membered heteroaryl optionally substituted by halo and comprising 1-to-2 heteroatoms independently selected from S and N.

An embodiment of the present invention provides compounds according to formula I as described herein, wherein R⁵ is H, arylalkyl, or arylalkyl substituted with halo.

An embodiment of the present invention provides compounds according to formula I as described herein, wherein R⁵ is H.

An embodiment of the present invention provides compounds according to formula I as described herein, wherein R⁶ is H.

An embodiment of the present invention provides compounds according to formula I as described herein, wherein
R¹ is alkoxy or haloalkoxy;
R² is halo, alkyl, alkoxy, haloalkyl, haloalkoxy, cyanoalkyl, cyanoalkoxy, or cyclopropyl optionally substituted with up to two substituents independently selected from cyano and halo;
R³ is H, alkoxy, or haloalkoxy;
R⁴ is cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with halo, alkyl, haloalkyl, alkoxy, haloalkoxy, or cyano;
R⁵ is H, halo, alkyl, aryl, heteroaryl, arylalkyl or heteroarylalkyl wherein aryl, heteroaryl, arylalkyl or heteroarylalkyl are optionally substituted with halo, alkyl, haloalkyl, alkoxy, haloalkoxy, or cyano;
R⁶ is H or halo;
and pharmaceutically acceptable salts.

An embodiment of the present invention provides compounds according to formula I as described herein, wherein
R¹ is alkoxy;
R² is haloalkyl or haloalkoxy;
R³ is alkoxy;
R⁴ is a 6-membered-aryl, or a 5-or-6-membered heteroaryl optionally substituted by halo and comprising 1-to-2 heteroatoms independently selected from S and N;
R⁵ is H;
R⁶ is H;
and pharmaceutically acceptable salts thereof.

An embodiment of the present invention provides compounds according to formula I as described herein, wherein
R¹ is alkoxy;
R² is haloalkyl, haloalkoxy, cyanoalkyl or cyclopropyl substituted with cyano;
R³ is H or alkoxy;
R⁴ is
   i. H;
   ii. Cyclobutyl;
   iii. 6-membered aryl;
   iv. A 5-membered heteroaryl comprising 1 S heteroatom and 1 N heteroatom;
   v. A 6-membered heteroaryl comprising 1 or 2 N heteroatoms;
   vi. A 6-member aryl substituted with halo;
   vii. A 5-membered heteroaryl comprising 1 S heteroatom and 1 N heteroatom substituted by alkyl;
   viii. A 6-membered heteroaryl comprising 1 N heteroatom substituted with alkyl, halo, or both alkyl and oxy.
R⁵ is H, arylalkyl, or arylalkyl substituted with halo;
R⁶ is H;
and pharmaceutically acceptable salts.

An embodiment of the present invention provides compounds according to formula I as described herein, wherein
R¹ is alkoxy;
R² is haloalkyl, haloalkoxy, cyanoalkyl or cyclopropyl substituted with cyano;
R³ is alkoxy;
R⁴ is
   i. 6-membered aryl;
   ii. A 5-membered heteroaryl comprising 1 S heteroatom and 1 N heteroatom;
   iii. A 6-membered heteroaryl comprising 1 N heteroatoms;
   iv. A 6-member aryl substituted with halo;
   v. A 5-membered heteroaryl comprising 1 S heteroatom and 1 N heteroatom substituted by alkyl;
   vi. A 6-membered heteroaryl comprising 1 N heteroatom substituted with alkyl or halo.
R⁵ is H;
R⁶ is H;
and pharmaceutically acceptable salts.

Particular examples of compounds of formula I as described herein are selected from
N-[5-(2,2-difluoroethoxy)-4,6-dimethoxy-pyrimidin-2-yl]-5-phenyl-1H-pyrrole-3-sulfonamide;
N-[5-(difluoromethoxy)-4,6-dimethoxy-pyrimidin-2-yl]-5-phenyl-1H-pyrrole-3-sulfonamide;
N-[5-(2-fluoroethoxy)-4,6-dimethoxy-pyrimidin-2-yl]-5-phenyl-1H-pyrrole-3-sulfonamide;
N-[5-(2,2-difluoroethoxy)-4,6-dimethoxy-pyrimidin-2-yl]-5-thiazol-2-yl-1H-pyrrole-3-sulfonamide;
N-[5-(difluoromethoxy)-4,6-dimethoxy-pyrimidin-2-yl]-5-(3-fluoro-2-pyridyl)-1H-pyrrole-3-sulfonamide;
N-[5-(2,2-difluoroethyl)-4,6-dimethoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-(3-fluoro-2-pyridyl)-1H-pyrrole-3-sulfonamide;
N-[5-(2,2-difluoroethoxy)-4,6-dimethoxy-pyrimidin-2-yl]-5-(3-fluoro-2-pyridyl)-1H-pyrrole-3-sulfonamide; and
   pharmaceutically acceptable salts thereof.

Other particular examples of compounds of formula I as described herein are selected from
N-[5-(2,2-difluoroethoxy)-4-methoxy-pyrimidin-2-yl]-5-thiazol-2-yl-TH-pyrrole-3-sulfonamide;
N-[5-(2-cyanoethyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-isothiazol-3-yl-1H-pyrrole-3-sulfonamide;
N-[5-(2,2-difluoroethyl)-4,6-dimethoxy-pyrimidin-2-yl]-5-phenyl-1H-pyrrole-3-sulfonamide;
N-[5-(2,2-difluoroethyl)-4,6-dimethoxy-pyrimidin-2-yl]-5-thiazol-2-yl-1H-pyrrole-3-sulfonamide;
N-[5-(difluoromethoxy)-4,6-dimethoxy-pyrimidin-2-yl]-5-thiazol-2-yl-1H-pyrrole-3-sulfonamide;
N-[5-(2,2-difluoroethyl)-4,6-dimethoxy-pyrimidin-2-yl]-5-thiazol-4-yl-1H-pyrrole-3-sulfonamide;
N-[5-(difluoromethoxy)-4,6-dimethoxy-pyrimidin-2-yl]-5-thiazol-4-yl-1H-pyrrole-3-sulfonamide;
N-[5-(2,2-difluoroethyl)-4,6-dimethoxy-pyrimidin-2-yl]-5-(2-pyridyl)-1H-pyrrole-3-sulfonamide;
N-[5-(difluoromethoxy)-4,6-dimethoxy-pyrimidin-2-yl]-5-(2-pyridyl)-1H-pyrrole-3-sulfonamide;
N-[5-(2,2-difluoroethoxy)-4,6-dimethoxy-pyrimidin-2-yl]-5-(2-pyridyl)-1H-pyrrole-3-sulfonamide;
N-[5-(2,2-difluoroethyl)-4,6-dimethoxy-pyrimidin-2-yl]-5-(4-methylthiazol-2-yl)-1H-pyrrole-3-sulfonamide;
4-benzyl-N-[5-(2,2-difluoroethyl)-4,6-dimethoxy-pyrimidin-2-yl]-1H-pyrrole-3-sulfonamide;
4-benzyl-N-[5-(difluoromethoxy)-4,6-dimethoxy-pyrimidin-2-yl]-1H-pyrrole-3-sulfonamide;
N-[5-(2-cyanoethyl)-4,6-dimethoxy-pyrimidin-2-yl]-5-(3-fluoro-2-pyridyl)-1H-pyrrole-3-sulfonamide;
N-[5-(2,2-difluoroethyl)-4-methoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-thiazol-4-yl-1H-pyrrole-3-sulfonamide;
N-[5-(2,2-difluoroethoxy)-4,6-dimethoxy-pyrimidin-2-yl]-5-thiazol-4-yl-1H-pyrrole-3-sulfonamide;
N-[5-(2-fluoroethoxy)-4,6-dimethoxy-pyrimidin-2-yl]-5-(2-pyridyl)-1H-pyrrole-3-sulfonamide;
4-benzyl-N-[5-(2-fluoroethoxy)-4,6-dimethoxy-pyrimidin-2-yl]-1H-pyrrole-3-sulfonamide;
4-[dideuterio-(3-fluorophenyl)methyl]-N-[5-(2,2-difluoroethoxy)-4,6-dimethoxy-pyrimidin-2-yl]-1H-pyrrole-3-sulfonamide;
4-benzyl-N-[5-(2,2-difluoroethoxy)-4,6-dimethoxy-pyrimidin-2-yl]-1H-pyrrole-3-sulfonamide;
N-[5-(2-fluoroethoxy)-4,6-dimethoxy-pyrimidin-2-yl]-5-(4-methylthiazol-2-yl)-1H-pyrrole-3-sulfonamide;
N-[5-(difluoromethoxy)-4,6-dimethoxy-pyrimidin-2-yl]-5-(4-methylthiazol-2-yl)-1H-pyrrole-3-sulfonamide;
N-[5-(2,2-difluoroethoxy)-4,6-dimethoxy-pyrimidin-2-yl]-5-(4-methylthiazol-2-yl)-1H-pyrrole-3-sulfonamide;
N-[5-(2,2-difluoroethyl)-4-methoxy-pyrimidin-2-yl]-5-thiazol-2-yl-1H-pyrrole-3-sulfonamide;
N-[5-(2-cyanocyclopropyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-thiazol-5-yl-1H-pyrrole-3-sulfonamide;
N-[5-(2,2-difluoroethyl)-4,6-dimethoxy-pyrimidin-2-yl]-5-(2-fluorophenyl)-1H-pyrrole-3-sulfonamide;
N-[5-(2-fluoroethoxy)-4,6-dimethoxy-pyrimidin-2-yl]-5-(2-fluorophenyl)-1H-pyrrole-3-sulfonamide;
N-[5-(difluoromethoxy)-4,6-dimethoxy-pyrimidin-2-yl]-5-(2-fluorophenyl)-1H-pyrrole-3-sulfonamide;
N-[5-(2,2-difluoroethoxy)-4,6-dimethoxy-pyrimidin-2-yl]-5-(2-fluorophenyl)-1H-pyrrole-3-sulfonamide;
N-[5-(2,2-difluoroethoxy)-4,6-dimethoxy-pyrimidin-2-yl]-5-(2-keto-1-methyl-3-pyridyl)-1H-pyrrole-3-sulfonamide;
N-[5-(2,2-difluoroethoxy)-4,6-dimethoxy-pyrimidin-2-yl]-5-(2-methylthiazol-5-yl)-1H-pyrrole-3-sulfonamide;
N-[5-(2,2-difluoroethoxy)-4,6-dimethoxy-pyrimidin-2-yl]-5-pyrimidin-2-yl-1H-pyrrole-3-sulfonamide;
N-[5-(2,2-difluoroethoxy)-4,6-dimethoxy-pyrimidin-2-yl]-5-(5-methylthiazol-2-yl)-1H-pyrrole-3-sulfonamide;
N-[5-(2,2-difluoroethoxy)-4,6-dimethoxy-pyrimidin-2-yl]-5-pyridazin-3-yl-1H-pyrrole-3-sulfonamide;
5-(3-chloro-2-pyridyl)-N-[5-(2,2-difluoroethoxy)-4,6-dimethoxy-pyrimidin-2-yl]-1H-pyrrole-3-sulfonamide;
N-[5-(2,2-difluoroethoxy)-4,6-dimethoxy-pyrimidin-2-yl]-5-pyrimidin-4-yl-1H-pyrrole-3-sulfonamide;
N-[5-(2,2-difluoroethoxy)-4,6-dimethoxy-pyrimidin-2-yl]-5-pyrazin-2-yl-1H-pyrrole-3-sulfonamide;
N-[5-(2,2-difluoroethoxy)-4,6-dimethoxy-pyrimidin-2-yl]-5-(3-methyl-2-pyridyl)-1H-pyrrole-3-sulfonamide;
5-cyclobutyl-N-[5-(difluoromethoxy)-4,6-dimethoxy-pyrimidin-2-yl]-1H-pyrrole-3-sulfonamide;
5-cyclobutyl-N-[5-(2,2-difluoroethoxy)-4,6-dimethoxy-pyrimidin-2-yl]-1H-pyrrole-3-sulfonamide; and
   pharmaceutically acceptable salts thereof.

Preferred examples of compounds of formula I as described herein are selected from
N-[5-(2-fluoroethoxy)-4,6-dimethoxy-pyrimidin-2-yl]-5-phenyl-1H-pyrrole-3-sulfonamide;
N-[5-(2,2-difluoroethoxy)-4,6-dimethoxy-pyrimidin-2-yl]-5-thiazol-2-yl-1H-pyrrole-3-sulfonamide;
N-[5-(difluoromethoxy)-4,6-dimethoxy-pyrimidin-2-yl]-5-(3-fluoro-2-pyridyl)-1H-pyrrole-3-sulfonamide;
N-[5-(2,2-difluoroethyl)-4,6-dimethoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-(3-fluoro-2-pyridyl)-1H-pyrrole-3-sulfonamide;
N-[5-(2,2-difluoroethoxy)-4,6-dimethoxy-pyrimidin-2-yl]-5-(3-fluoro-2-pyridyl)-1H-pyrrole-3-sulfonamide;
N-[5-(2-cyanoethyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-isothiazol-3-yl-1H-pyrrole-3-sulfonamide;
N-[5-(2,2-difluoroethyl)-4,6-dimethoxy-pyrimidin-2-yl]-5-phenyl-1H-pyrrole-3-sulfonamide;
N-[5-(2,2-difluoroethyl)-4,6-dimethoxy-pyrimidin-2-yl]-5-thiazol-2-yl-1H-pyrrole-3-sulfonamide;
N-[5-(2,2-difluoroethyl)-4,6-dimethoxy-pyrimidin-2-yl]-5-(2-pyridyl)-1H-pyrrole-3-sulfonamide;
N-[5-(2,2-difluoroethoxy)-4,6-dimethoxy-pyrimidin-2-yl]-5-(2-pyridyl)-1H-pyrrole-3-sulfonamide;
N-[5-(2,2-difluoroethyl)-4,6-dimethoxy-pyrimidin-2-yl]-5-(4-methylthiazol-2-yl)-1H-pyrrole-3-sulfonamide;
N-[5-(2-cyanoethyl)-4,6-dimethoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-(2-pyridyl)-1H-pyrrole-3-sulfonamide;
N-[5-(2-fluoroethoxy)-4,6-dimethoxy-pyrimidin-2-yl]-5-(4-methylthiazol-2-yl)-1H-pyrrole-3-sulfonamide;
N-[5-(2-cyanocyclopropyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-thiazol-5-yl-1H-pyrrole-3-sulfonamide;
N-[5-(2,2-difluoroethyl)-4,6-dimethoxy-pyrimidin-2-yl]-5-(2-fluorophenyl)-1H-pyrrole-3-sulfonamide;
N-[5-(2-fluoroethoxy)-4,6-dimethoxy-pyrimidin-2-yl]-5-(2-fluorophenyl)-1H-pyrrole-3-sulfonamide;
5-(3-chloro-2-pyridyl)-N-[5-(2,2-difluoroethoxy)-4,6-dimethoxy-pyrimidin-2-yl]-1H-pyrrole-3-sulfonamide;
N-[5-(2,2-difluoroethoxy)-4,6-dimethoxy-pyrimidin-2-yl]-5-(3-methyl-2-pyridyl)-1H-pyrrole-3-sulfonamide; and
   pharmaceutically acceptable salts thereof.

Processes for the manufacture of compounds of formula I as described herein are an object of the invention.

The present compounds of formula I and their pharmaceutically acceptable salts can be prepared by methods known in the art, for example, by processes described below, which process comprises reacting a compound of formula III with a compound of formula II in the presence of a base selected from N-ethyldiisopropylamine, pyridine, potassium phosphate or sodium hydride, to provide a compound of formula I,

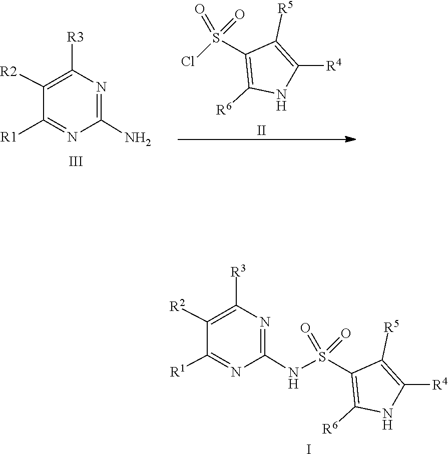

wherein R¹, R², R³, R³, R⁴, R⁵ and R⁶ are as described above.

Alternatively, the present compounds of formula I and their pharmaceutically acceptable salts can be prepared by methods known in the art, for example, by processes described below, which process comprises reacting protected sulfonylchloride IV with amines III in the presence of a base like N-ethyldiisopropylamine or pyridine to form compound V which is then treated with a base such as potassium hydroxide or sodium hydroxide in ethanol or methanol to give the desired compound I,

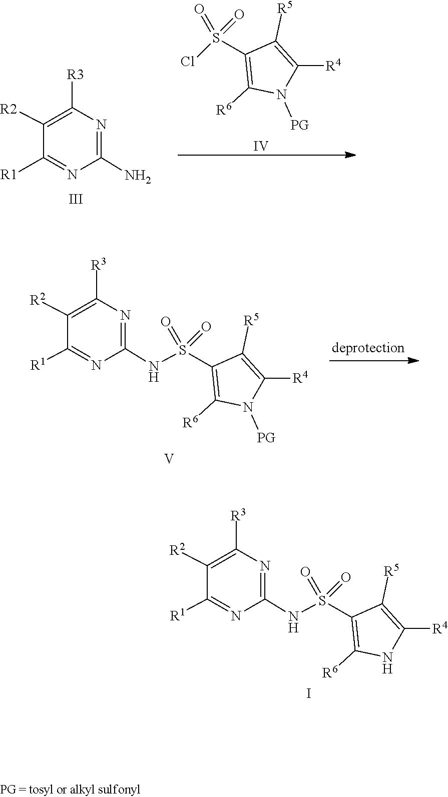

wherein R¹, R², R³, R³, R⁴, R⁵ and R⁶ are as described above.

Alternatively, the present compounds of formula I and their pharmaceutically acceptable salts can be prepared by methods known in the art, for example, by processes described below, which process comprises amines III reacted with protected sulfonylchloride VI in the presence of a base like N-ethyldiisopropylamine or pyridine to form compound VII which is then treated with a base such as potassium hydroxide or sodium hydroxide in ethanol or methanol to give intermediate VIII. This compound can be brominated using N-bromosuccinimide to form intermediate IX which is further reacted with bis(pinacolato)diboron in presence of a base such as potassium acetate and a suitable palladium catalyst such as [1,1′-bis(diphenylphosphino)ferrocene]palladium(II) to form intermediate X. Reaction of X with an heteroaryl-halide XI in presence of a base such as potassium phosphate, potassium carbonate or cesium carbonate and a palladium source such as tetrakis(triphenylphosphine)-palladium(0), XPhos Pd G4, [1,1′-bis(diphenylphosphino)ferrocene]dichloropalladium(II) or the like yields the desired compound I.

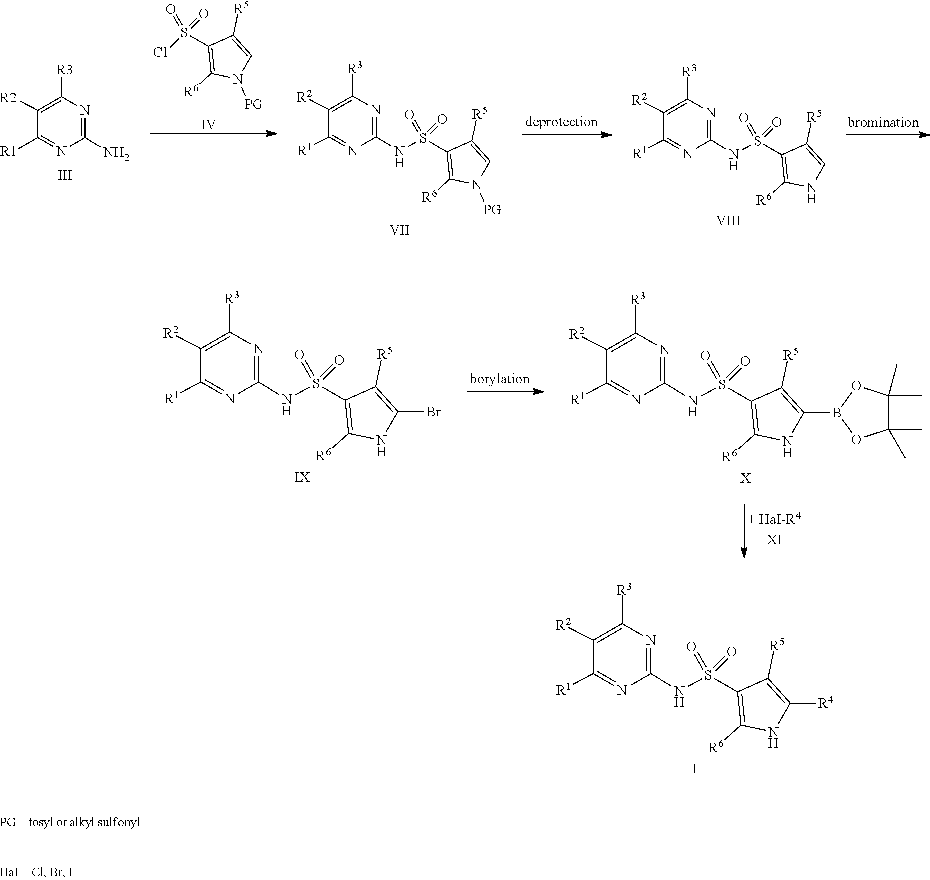

wherein R¹, R², R³, R³, R⁴, R⁵ and R⁶ are as described above.

### General Synthetic Schemes

The compounds of formula I may be prepared in accordance with the process variant described above and with the following scheme 1. The starting materials are commercially available or may be prepared in accordance with known methods.

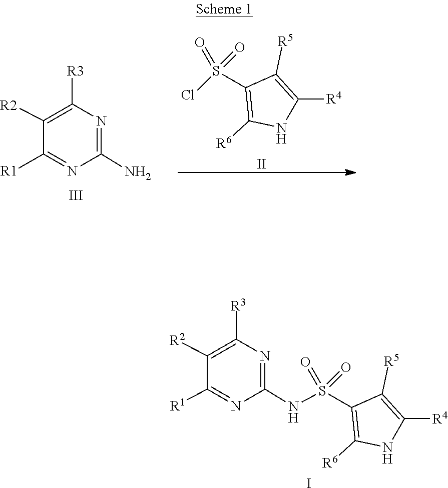

Compounds of general formula I can be prepared by reacting sulfonylchloride II with amines III in the presence of a base like N-ethyldiisopropylamine, pyridine, potassium phosphate or sodium hydride. The starting materials are commercially available or may be prepared in accordance with known methods.

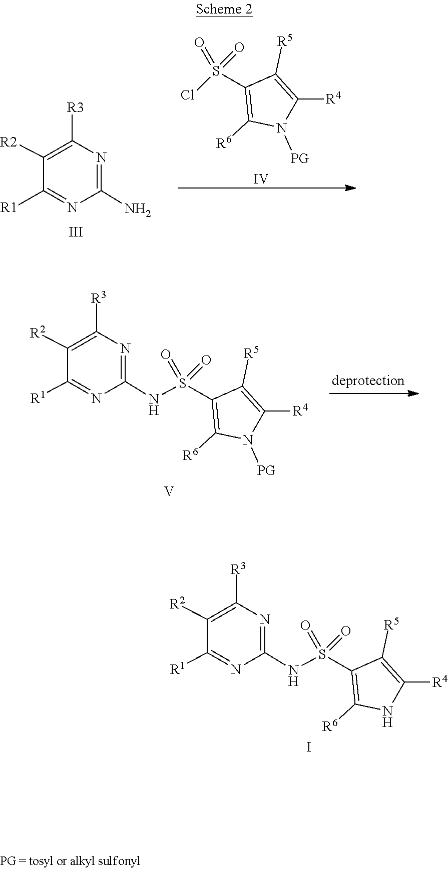

Furthermore, compounds of general formula I can be prepared by reacting protected sulfonylchloride IV with amines III in the presence of a base like N-ethyldiisopropylamine or pyridine to form compound V which is then treated with a base such as potassium hydroxide or sodium hydroxide in ethanol or methanol to give the desired compound I.

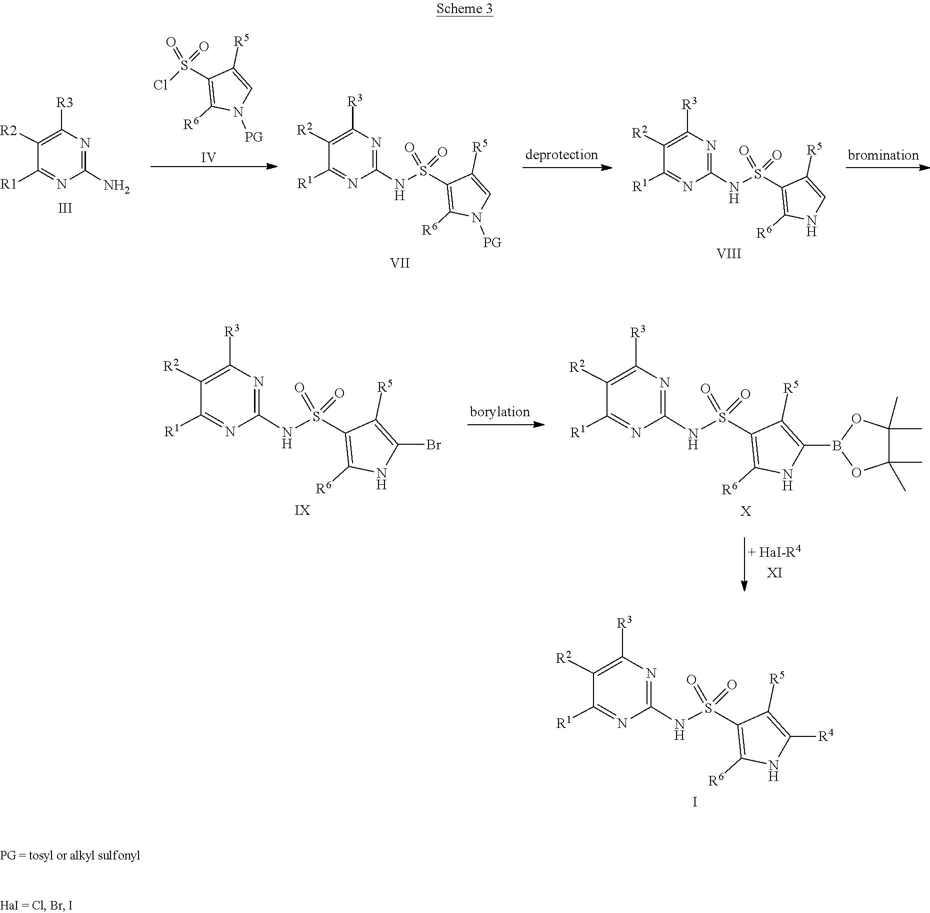

In addition, amines III can be reacted with protected sulfonylchloride VI in the presence of a base like N-ethyldiisopropylamine or pyridine to form compound VII which is then treated with a base such as potassium hydroxide or sodium hydroxide in ethanol or methanol to give intermediate VIII. This compound can be brominated using N-bromosuccinimide to form intermediate IX which is further reacted with bis(pinacolato)diboron in presence of a base such as potassium acetate and a suitable palladium catalyst such as [1,1′-bis(diphenylphosphino)ferrocene]palladium(II) to form intermediate X. Reaction of X with an heteroaryl-halide XI in presence of a base such as potassium phosphate, potassium carbonate or cesium carbonate and a palladium source such as tetrakis(triphenylphosphine)-palladium(0), XPhos Pd G4, [1,1′-bis(diphenylphosphino)ferrocene]dichloropalladium(II) or the like yields the desired compound I.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

The invention also relates in particular to:
A compound of formula I for use as therapeutically active substance;
A compound of formula I for use in the treatment of a disease modulated by GPR17;
Likewise an object of the present invention is a pharmaceutical composition comprising a compound according to formula I as described herein and a therapeutically inert carrier.

The use of a compound of formula I for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the treatment or prophylaxis of multiple sclerosis, Alzheimer's disease, Parkinson's disease, or Huntington's disease.

A particular embodiment of the invention is the use of a compound of formula I for the treatment or prophylaxis of multiple sclerosis.

The use of a compound of formula I for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the preparation of a medicament for the treatment or prophylaxis of multiple sclerosis, Alzheimer's disease, Parkinson's disease, or Huntington's disease.

A particular embodiment of the invention is the use of a compound of formula I for the preparation of a medicament for the treatment or prophylaxis of multiple sclerosis.

A compound according to formula I for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is a compound of formula I for use in the treatment or prophylaxis of multiple sclerosis, Alzheimer's disease, Parkinson's disease, or Huntington's disease.

A particular embodiment of the invention is a compound according to formula I for use in the treatment or prophylaxis of multiple sclerosis.

A method for the treatment or propylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity, which method comprises administering an effective amount of a compound of formula I to a patient in need thereof.

An embodiment of the present invention is a method for the treatment or prophylaxis of multiple sclerosis, Alzheimer's disease, Parkinson's disease, or Huntington's disease, which method comprises administering an effective amount of a compound of formula I to a patient in need thereof.

A particular embodiment of the invention is a method for the treatment or prophylaxis of multiple sclerosis, which method comprises administering an effective amount of a compound of formula I to a patient in need thereof.

Also an embodiment of the present invention provides compounds of formula I as described herein, when manufactured according to any one of the described processes.

### Assay Procedures

GPR17 cAMP Assay Protocol:

CHO-K1 cells stably expressing vector containing untagged human GPR17 short isoform (Roche) were cultured at 37° C./5% CO2 in DMEM (Dulbecco's Modified Eagle Medium):F-12 (1:1) supplemented with 10% foetal bovine serum and 400 μg/ml Geneticin.

Changes in intracellular cyclic adenosine monophosphate (cAMP) levels were quantified using the Nano-TRF Detection Assay kit (Roche Diagnostics, Cat. No. 05214386001). This assay allows for direct cAMP quantification in a homogeneous solution. cAMP is detected based on time-resolved fluorescence energy transfer (TR-FRET) and competitive binding of ruthenylated cAMP and endogenous cAMP to an anti-cAMP monoclonal antibody labeled with AlexaFluor-700. The Ruthenium complex serves as the FRET donor and transfers energy to AlexaFluor-700. The FRET signal is inversely proportional to the cAMP concentration.

CHO-GPR17S cells were detached with Accutase and resuspended in assay buffer consisting of Hank's Balanced Salt Solution (HBSS), 10 mM HEPES (4-(2-hydroxyethyl) piperazine-1-ethanesulfonic acid solution) and 0.1% bovine serum albumin (pH 7.4). The cells were seeded in black 384-well plates (Corning) at a density of 10'000 cells/20 μl assay buffer until the addition of compounds.

Test antagonist compounds were serially diluted in dimethyl sulfoxide (DMSO) and spotted in 384-well plates. The compounds were then diluted in HBSS buffer supplemented with an EC80 concentration of MDL29,951 (3-(2-Carboxy-4,6-dichloroindol-3-yl)propionic acid) (GPR17 agonist) plus 3-Isobutyl-1-methylxanthine (IBMX) (0.5 mM final concentration) and added to the cells at room temperature. Forskolin (15 μM final concentration) was added 5 minutes after the test compounds and the cells were incubated at room temperature for 30 minutes. The assay was stopped by adding cAMP detection mix (containing detergents for cell lysis) for 90 minutes at room temperature.

Cellular cAMP was measured using a Paradigm reader (Molecular Devices). The raw data was used to calculate the FRET signal based on the assay's P-factor as per cAMP kit instructions. The data was normalized to the maximal activity of a reference antagonist and dose response curves were fitted to the percent activity of the test compounds using a sigmoidal dose response model (Genedata Screener).

Results in the hGPR17 cAMP assay are provided for compounds of formula I in Table 1

TABLE 1

| Example | hGPR17 cAMP |
|---|---|
| number | IC50 [μM] |
| 1 | 0.476 |
| 2 | 0.478 |
| 3 | 0.073 |
| 4 | 0.500 |
| 5 | 0.325 |
| 6 | 0.107 |
| 7 | 0.085 |
| 8 | 0.099 |
| 9 | 0.697 |
| 10 | 0.069 |
| 11 | 0.142 |
| 12 | 0.169 |
| 13 | 0.098 |
| 14 | 0.256 |
| 15 | 0.661 |
| 16 | 2.266 |
| 17 | 0.133 |
| 18 | 0.344 |
| 19 | 0.182 |
| 20 | 0.198 |
| 21 | 0.635 |
| 22 | 2.255 |
| 23 | 0.045 |
| 24 | 0.331 |
| 25 | 0.916 |
| 26 | 0.724 |
| 27 | 0.127 |
| 28 | 1.078 |
| 29 | 0.964 |
| 30 | 1.863 |
| 31 | 0.222 |
| 32 | 0.523 |
| 33 | 0.283 |
| 34 | 0.457 |
| 35 | 0.170 |
| 36 | 0.111 |
| 37 | 0.130 |
| 38 | 0.124 |
| 39 | 0.354 |
| 40 | 0.540 |
| 41 | 1.424 |
| 42 | 1.988 |
| 43 | 1.248 |
| 44 | 2.669 |
| 45 | 0.253 |
| 46 | 0.077 |
| 47 | 1.608 |
| 48 | 1.316 |
| 49 | 0.122 |
| 50 | 0.748 |
| 51 | 0.304 |

### Microsome Clearance Assay Protocol:

incubations at a test compound of 1 mM in microsomes (0.5 mg/mL) plus cofactor NADPH are performed in 96 well plates at 37° C. on a TECAN (Tecan Group Ltd, Switzerland) automated liquid handling system. The final concentration of the test compound in the incubation is 1 microM. After a 10 minutes preincubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standards. Samples are then cooled and centrifuged before analysis of the supernatant by LC-MS/MS.

Reference examples RE-A, RE-B, RE-C, RE-D, RE-E, and RE-F have been prepared as described herein.

Reference compounds were tested against exemplified compounds for their Microsome Clearance. The results are shown in Table 2 below.

TABLE 2
Microsomal Clearance

| | Microsomal Clearance |
|---|---|
| | human, mouse, rat |
| Compound | (μL/min/mg protein) |
| Example 1 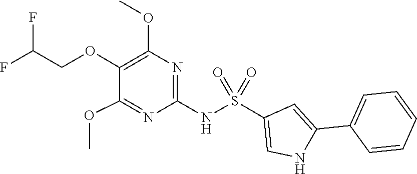 [SMILES: COc1nc(NS(=O)(=O)c2c[nH]c(-c3ccccc3)c2)nc(OC)c1OCC(F)F] | 10, 21, 81 |
| Example 7 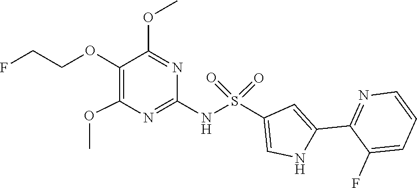 [SMILES: COc1nc(NS(=O)(=O)c2c[nH]c(-c3ncccc3F)c2)nc(OC)c1OCCF] | 10, 12, 32 |
| Example 14 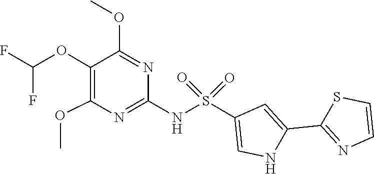 [SMILES: COc1nc(NS(=O)(=O)c2c[nH]c(-c3nccs3)c2)nc(OC)c1OC(F)F] | 10, 10, 50 |
| RE-A 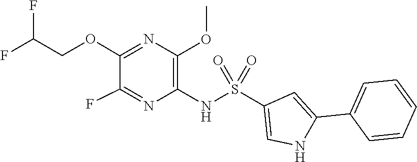 | 21, 136, 489 |
| RE-B 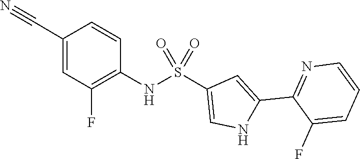 | 253, 43, 79 |
| RE-C 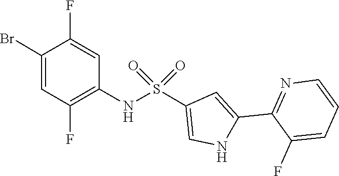 | 107, 238, 582 |
| RE-D 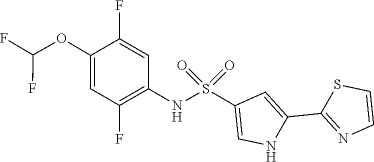 | 10, 35, 207 |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Intermediates A1-A3

The intermediates A1-A3 are known and have been prepared following procedures described in WO2022254027:

| Intermediate | | |
|---|---|---|
| number | Structure | Name (CAS number) |
| A1 | 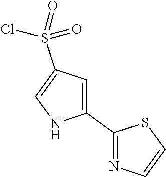 [SMILES: O=S(=O)(Cl)c1c[nH]c(-c2nccs2)c1] | 5-thiazol-2-yl-1H-pyrrole-3- sulfonyl chloride |
| A2 | 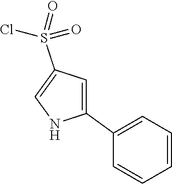 [SMILES: O=S(=O)(Cl)c1c[nH]c(-c2ccccc2)c1] | 5-phenyl-1H-pyrrole-3- sulfonyl chloride (2877693- 34-8) |
| A3 | 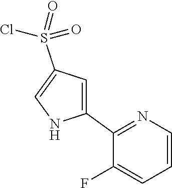 [SMILES: O=S(=O)(Cl)c1c[nH]c(-c2ncccc2F)c1] | 5-(3-fluoro-2-pyridyl)-1H- pyrrole-3-sulfonyl chloride (2877693-76-8) |

### Intermediates A4-A13

The intermediates A4-A7 are known and have been prepared following procedures described in WO2022254027:

| Intermediate | | |
|---|---|---|
| number | Structure | Name (CAS number) |
| A4 | 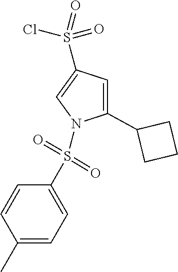 [SMILES: Cc1ccc(S(=O)(=O)n2cc(S(=O)(=O)Cl)cc2C2CCC2)cc1] | 5-cyclobutyl-1-(p- tolylsulfonyl)pyrrole-3- sulfonyl chloride (2877694-06-7) |
| A5 | 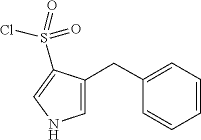 [SMILES: O=S(=O)(Cl)c1c[nH]cc1Cc1ccccc1] | 4-benzyl-1H-pyrrole-3- sulfonyl chloride (2877694-28-3) |
| A6 | 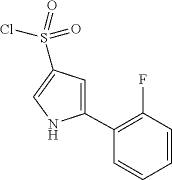 [SMILES: O=S(=O)(Cl)c1c[nH]c(-c2ccccc2F)c1] | 5-(2-fluorophenyl)-1H- pyrrole-3-sulfonyl chloride (2877693-62-2) |
| A7 | 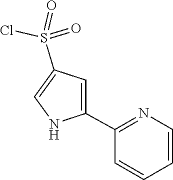 [SMILES: O=S(=O)(Cl)c1c[nH]c(-c2ccccn2)c1] | 5-(2-pyridyl)-1H-pyrrole-3- sulfonyl chloride (2877693-77-9) |

### Intermediate A8: 5-(4-methylthiazol-2-yl)-1H-pyrrole-3-sulfonyl chloride

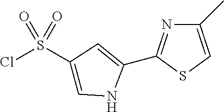

### Step 1: tert-butyl 2-(4-methylthiazol-2-yl)pyrrole-1-carboxylate

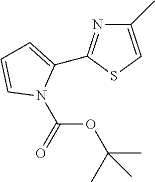 [SMILES: Cc1csc(-c2cccn2C(=O)OC(C)(C)C)n1]

A solution of (1-tert-butoxycarbonylpyrrol-2-yl)boronic acid (CAS 135884-31-0; 1.0 g, 4.64 mmol), 2-bromo-4-methyl-thiazole (861 mg, 521 uL, 4.64 mmol), potassium carbonate (1.93 g, 13.9 mmol) and tetrakis(triphenylphosphine)palladium(0) (537 mg, 0.464 mmol, 0.10 eq) in 1,4-dioxane (20 mL) and water (2 mL) was heated to 80° C. and stirred for 15 h. The reaction mixture was poured into brine and extracted twice with ethyl acetate The organic layers were dried over Na2SO4, filtered and concentrated in vacuo. The residue was purified by flash chromatography (silica gel, 0% to 30% ethyl acetate in heptane) to provide tert-butyl 2-(4-methylthiazol-2-yl)pyrrole-1-carboxylate (784 mg, 62% yield) as colorless oil. MS (ESI) m/z: 208.6 [M+H-butene]⁺.

### Step 2: 5-(4-methylthiazol-2-yl)-1H-pyrrole-3-sulfonyl chloride

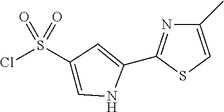

A solution of tert-butyl 2-(4-methylthiazol-2-yl)pyrrole-1-carboxylate (732 mg, 2.69 mmol) in acetonitrile (25 mL) was added chlorosulfonic acid (814 mg, 468 uL, 6.98 mmol) dropwise at 0° C. The reaction mixture was stirred at room temperature for 2 h. Then phosphorus oxychloride (1.24 g, 751 uL, 8.06 mmol) was added and the reaction mixture was heated to 60° C. and stirred for 15 h. The reaction mixture was poured into ice/ethyl acetate and extracted twice with ethyl acetate. The organic layers were washed with brine, dried over Na2SO4, filtered and concentrated in vacuo to provide 5-(4-methylthiazol-2-yl)-1H-pyrrole-3-sulfonyl chloride (542 mg, 77% yield) as brown viscous oil. MS (ESI) m/z: 263.0 [M+H]⁺.

### Intermediate A9: 5-thiazol-4-yl-1H-pyrrole-3-sulfonyl chloride

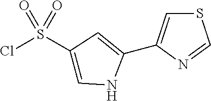 [SMILES: O=S(=O)(Cl)c1c[nH]c(-c2cscn2)c1]

### Step 1: tert-butyl 2-thiazol-4-ylpyrrole-1-carboxylate

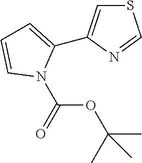 [SMILES: CC(C)(C)OC(=O)n1cccc1-c1cscn1]

To a solution of (1-tert-butoxycarbonylpyrrol-2-yl)boronic acid (CAS 135884-31-0; 2.0 g, 9.48 mmol) and 4-bromothiazole (1.71 g, 930 uL, 10.43 mmol) in 1,4-dioxane (45 mL) and water (4 mL) was added potassium carbonate (3.93 g, 28.4 mmol). The reaction mixture was degassed with argon then tetrakis(triphenylphosphine)palladium(0) (1.1 g, 948 umol, 0.10 eq) was added. The reaction mixture was stirred at 80° C. overnight. After cooling to room temperature the reaction mixture was filtered over decalite and washed with ethyl acetate. The filtrate was poured into water and extracted with ethyl acetate twice. The combined organic layers were washed with brine, dried over Na2SO4, filtered and concentrated in vacuo. The residue was purified by flash chromatography (silica gel, 0% to 30% ethyl acetate in heptane) to afford tert-butyl 2-thiazol-4-ylpyrrole-1-carboxylate (1.73 g, 72% yield) as colorless liquid. MS (ESI) m/z: 251.1 [M+H]⁺.

### Step 2: 5-thiazol-4-yl-1H-pyrrole-3-sulfonyl chloride

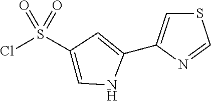 [SMILES: O=S(=O)(Cl)c1c[nH]c(-c2cscn2)c1]

To a solution of tert-butyl 2-thiazol-4-ylpyrrole-1-carboxylate (1.11 g, 4.43 mmol) in acetonitrile (10 mL) was added 4 M HCl in dioxane (13.3 g, 11.1 mL, 44.3 mmol) and the mixture was stirred 2 h at 60° C. After cooling to room temperature the reaction mixture was poured into ice-cold NaHCO3 solution and extracted with ethyl acetate twice. The combined organic layers were washed with brine, dried over Na2SO4, filtered and concentrated in vacuo. The residue was dissolved in acetonitrile (15 mL) and the solution was cooled to 0° C. Chlorosulfonic acid (517 mg, 297 uL, 4.43 mmol) was added. The ice bath was removed and the reaction mixture was stirred 4 h at room temperature. Then phosphorus oxychloride (2.04 g, 1.24 mL, 13.3 mmol) was added and the reaction mixture was stirred at 60° C. overnight. The reaction mixture was poured into cold water and extracted with ethyl acetate twice. The combined organic layers were washed with brine, dried over Na2SO4, filtered and concentrated in vacuo. The crude material was purified by flash chromatography (silica gel, 0% to 50% ethyl acetate in heptane) to afford 5-thiazol-4-yl-1H-pyrrole-3-sulfonyl chloride (418 mg, 38% yield, second eluting regioisomer) as white solid. MS (ESI) m/z: 246.9 [M−H]⁻.

### Intermediate A10: 5-thiazol-5-yl-1H-pyrrole-3-sulfonyl chloride

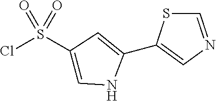 [SMILES: O=S(=O)(Cl)c1c[nH]c(-c2cncs2)c1]

The title compound was prepared in analogy to Intermediate A8 from 5-bromothiazole instead of 2-bromo-4-methyl-thiazole in Step 1) as light grey powder. MS (ESI) m/z: 249.0 [M+H]⁺.

### Intermediate A11: 5-isothiazol-3-yl-1H-pyrrole-3-sulfonyl chloride

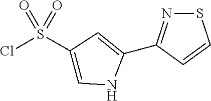 [SMILES: O=S(=O)(Cl)c1c[nH]c(-c2ccsn2)c1]

The title compound was prepared in analogy to Intermediate A8 from 3-bromoisothiazole instead of 2-bromo-4-methyl-thiazole in Step 1) as grey solid. MS (ESI) m/z: 246.9 [M−H]⁻.

### Intermediate A12: 5-(1-methyl-2-oxo-3-pyridyl)-1H-pyrrole-3-sulfonyl chloride

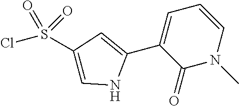 [SMILES: Cn1cccc(-c2cc(S(=O)(=O)Cl)c[nH]2)c1=O]

The title compound was prepared in analogy to Intermediate A8 from 3-bromo-1-methyl-2-pyridone instead of 2-bromo-4-methyl-thiazole in Step 1) as dark green solid. MS (ESI) m/z: 273.0 [M+H]⁺.

### Intermediate A13: 4-[dideuterio-(3-fluorophenyl)methyl]-1H-pyrrole-3-sulfonyl chloride

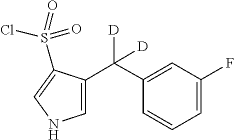 [SMILES: [2H]C([2H])(c1cccc(F)c1)c1c[nH]cc1S(=O)(=O)Cl]

### Step 1: 3-[dideuterio-(3-fluorophenyl)methyl]-1H-pyrrole

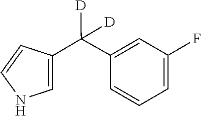 [SMILES: [2H]C([2H])(c1cc[nH]c1)c1cccc(F)c1]

A mixture of 2-[dideuterio-(3-fluorophenyl)methyl]-1-(p-tolylsulfonyl)pyrrole (CAS 2877694-47-6, see WO2022254027; 6.9 g, 20.82 mmol), sodium hydroxide (4N in water, 26.0 mL, 104.1 mmol) in methanol (45 mL) was stirred at 80° C. for 12 h. The reaction mixture was diluted with water 100 mL and extracted with ethyl acetate (80 mL×3). The combined organic layers were washed with saturated sodium chloride solution 30 mL, dried over Na2SO4, filtered and concentrated under reduced pressure. The residue was purified by flash silica gel chromatography (silica gel, 0-20% ethyl acetate in petroleum ether) to give 3-[dideuterio-(3-fluorophenyl)methyl]-1H-pyrrole (3.64 g, 20.6 mmol, 95% yield) as a light yellow oil. MS (ESI) m/z: 178.1 [M+H]⁺.

### Step 2: 4-[dideuterio-(3-fluorophenyl)methyl]-1H-pyrrole-3-sulfonyl chloride

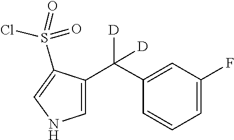 [SMILES: [2H]C([2H])(c1cccc(F)c1)c1c[nH]cc1S(=O)(=O)Cl]

A stirred solution of 3-[dideuterio-(3-fluorophenyl)methyl]-1H-pyrrole (200 mg, 1.08 mmol) in acetonitrile (6 mL) was cooled to 0° C. Chlorosulfonic acid (133 mg, 76 uL, 1.14 mmol) was added dropwise and the reaction mixture was stirred at 0° C. for 90 min. Then phosphorus oxychloride (664 mg, 404 uL, 4.33 mmol) was added. The reaction mixture was warmed to 70° C. and stirred for 16 h. The reaction mixture was allowed to cool to room temperature, poured into ice/ethyl acetate and extracted three times with ethyl acetate. The organic layers were washed with brine, dried with sodium sulfate, filtered and concentrated in vacuo to provide the title compound (364 mg, 51% yield) as dark brown oil. MS (ESI) m/z: 274.0 [M−H]⁻.

### Intermediates B1-B4

The intermediates B1-B4 are known and have been prepared following procedures described in WO2022180136:

| Intermediate | | |
|---|---|---|
| number | Structure | Name (CAS number) |
| B1 | 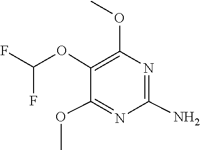 | 5-(difluoromethoxy)-4,6- dimethoxy-pyrimidin-2-amine (2827058-56-8) |
| B2 | 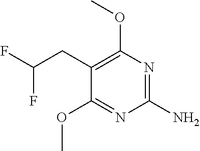 | 5-(2,2-difluoroethyl)-4,6- dimethoxy-pyrimidin-2-amine (2827058-75-1) |
| B3 | 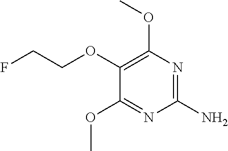 | 5-(2-fluoroethoxy)-4,6- dimethoxy-pyrimidin-2-amine (2827058-58-0) |
| B4 | 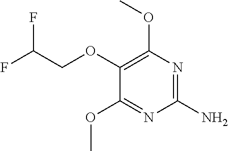 [SMILES: COc1nc(N)nc(OC)c1OCC(F)F] | 5-(2,2-difluoroethoxy)-4,6- dimethoxy-pyrimidin-2-amine (2827058-60-4) |

### Intermediates B5-B8

The intermediates B5-B7 are known and have been prepared following procedures described in WO2022180136:

| Intermediate | | |
|---|---|---|
| number | Structure | Name (CAS number) |
| B5 | 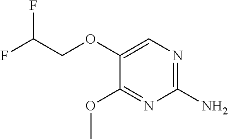 | 5-(2,2-difluoroethoxy)-4- methoxy-pyrimidin-2-amine (2827057-91-8) |
| B6 | 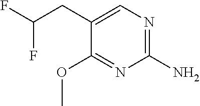 | 5-(2,2-difluoroethyl)-4- methoxy-pyrimidin-2-amine (2827058-18-2) |
| B7 | 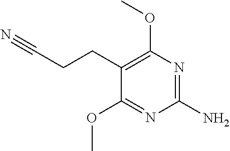 | 3-(2-amino-4,6-dimethoxy- pyrimidin-5-yl)propanenitrile (2117776-96-0) |

### Intermediate B8: 2-(2-amino-4,6-dimethoxy-pyrimidin-5-yl)cyclopropanecarbonitrile

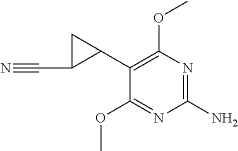 [SMILES: COc1nc(N)nc(OC)c1C1CC1C#N]

### Step 1: N,N-bis[(2,4-dimethoxyphenyl)methyl]-4,6-dimethoxy-pyrimidin-2-amine

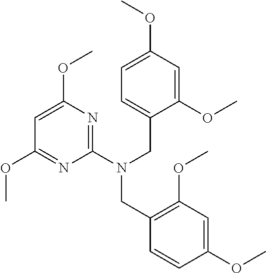 [SMILES: COc1ccc(CN(Cc2ccc(OC)cc2OC)c2nc(OC)cc(OC)n2)c(OC)c1]

To a solution of 2-chloro-4,6-dimethoxypyrimidine (5.0 g, 28.6 mmol) and 1-(2,4-dimethoxyphenyl)-N-[(2,4-dimethoxyphenyl)methyl]methanamine (10.9 g, 34.37 mmol) in N-methylpyrrolidone (100 ml) was added cesium carbonate (18.7 g, 57.3 mmol) and the reaction mixture was stirred at 120° C. for 16 h. The mixture was diluted with water (500 ml) and extracted with ethyl acetate (100 mL×2). The combined organic layers were washed with brine (100 mL×2), dried over Na2SO4, filtered and concentrated under reduced pressure. The residue was purified by column chromatography (silica gel, petroleum ether/ethyl acetate=1:0 to 5:1) to give N-bis[(2,4-dimethoxyphenyl)methyl]-4,6-dimethoxy-pyrimidin-2-amine (12.0 g, 89% yield) as a white solid. MS (ESI) m/z: 456.3 [M+H]⁺

### Step 2: N,N-bis[(2,4-dimethoxyphenyl)methyl]-5-iodo-4,6-dimethoxy-pyrimidin-2-amine

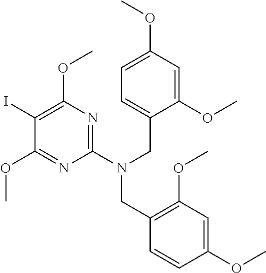 [SMILES: COc1ccc(CN(Cc2ccc(OC)cc2OC)c2nc(OC)c(I)c(OC)n2)c(OC)c1]

To a solution of N,N-bis[(2,4-dimethoxyphenyl)methyl]-4,6-dimethoxy-pyrimidin-2-amine (12.0 g, 26.3 mmol) in acetonitrile (150 mL) was added N-iodosuccinimide (7.11 g, 31.6 mmol) in portions at 20° C. and the mixture was stirred at 20° C. for 2 h. The reaction mixture was quenched by pouring into saturated sodium hydrogencarbonate solution (300 mL) and extracted with ethyl acetate (100 mL×2). The combined organic layers were washed with brine (100 mL×2), dried over sodium sulfate, filtered and concentrated under reduced pressure. The residue was purified by column chromatography (silica gel, petroleum ether/ethyl acetate=1:0 to 3:1) to give N,N-bis[(2,4-dimethoxyphenyl)methyl]-5-iodo-4,6-dimethoxy-pyrimidin-2-amine (12.0 g, 78% yield) as a yellow solid. MS (ESI) m/z: 582.2 [M+H]⁺

### Step 3: (E)-3-[2-[bis[(2,4-dimethoxyphenyl)methyl]amino]-4,6-dimethoxy-pyrimidin-5-yl]prop-2-enenitrile

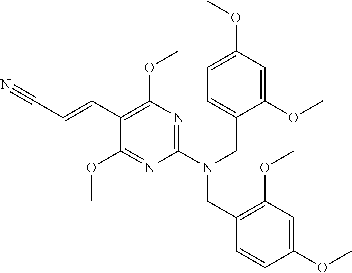 [SMILES: COc1ccc(CN(Cc2ccc(OC)cc2OC)c2nc(OC)c(/C=C/C#N)c(OC)n2)c(OC)c1]

To a mixture of N,N-bis[(2,4-dimethoxyphenyl)methyl]-5-iodo-4,6-dimethoxy-pyrimidin-2-amine (9.0 g, 15.5 mmol) and acrylonitrile (6.0 ml, 90.5 mmol) in 1,4-dioxane (90 mL) was added cesium carbonate (10.8 g, 33.1 mmol) and bis(triphenylphosphine)palladium chloride (681 mg, 1.55 mmol) under nitrogen atmosphere and the reaction mixture was stirred in a sealed tube at 100° C. for 16 h. The mixture was concentrated under reduced pressure.

The residue was purified by column chromatography (silica gel, petroleum ether/ethyl acetate=1:0 to 5:1) to give (E)-3-[2-[bis[(2,4-dimethoxyphenyl)methyl]amino]-4,6-dimethoxy-pyrimidin-5-yl]prop-2-enenitrile (7.5 g, 96% yield) as a brown solid. MS (ESI) m/z: 507.3 [M+H]⁺

### Step 4: 2-[2-[bis[(2,4-dimethoxyphenyl)methyl]amino]-4,6-dimethoxy-pyrimidin-5-yl]cyclopropanecarbonitrile

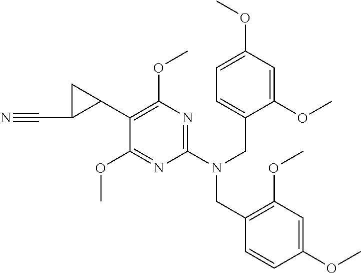 [SMILES: COc1ccc(CN(Cc2ccc(OC)cc2OC)c2nc(OC)c(C3CC3C#N)c(OC)n2)c(OC)c1]

To a mixture of trimethylsulfoxonium iodide (6.52 g, 29.6 mmol) in dimethylsulfoxide (50 ml) was added sodium hydride (60% in mineral oil, 1.18 g, 29.6 mmol) in portions at 30° C. and the mixture was stirred at 30° C. for 0.5 h. The above mixture was added to a mixture of (E)-3-[2-[bis[(2,4-dimethoxyphenyl)methyl]amino]-4,6-dimethoxy-pyrimidin-5-yl]prop-2-enenitrile (5.0 g, 9.87 mmol) in dimethylsulfoxide (50 mL) at 30° C. and the mixture was stirred at 30° C. for 16 hr. The reaction mixture was diluted with water (300 ml) and extracted with ethyl acetate (100 mL×2). The combined organic layers were washed with brine (100 mL×2), dried over sodium sulfate, filtered and concentrated under reduced pressure. The residue was purified by column chromatography (silica gel, petroleum ether/ethyl acetate=1:0 to 3:1) to give 2-[2-[bis[(2,4-dimethoxyphenyl)methyl]amino]-4,6-dimethoxy-pyrimidin-5-yl]cyclopropanecarbonitrile (3.0 g, 54% yield) as yellow oil. MS (ESI) m/z: 521.3 [M+H]⁺

### Step 5: 2-(2-amino-4,6-dimethoxy-pyrimidin-5-yl)cyclopropanecarbonitrile

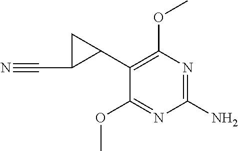 [SMILES: COc1nc(N)nc(OC)c1C1CC1C#N]

To a solution of 2-[2-[bis[(2,4-dimethoxyphenyl)methyl]amino]-4,6-dimethoxy-pyrimidin-5-yl]cyclopropanecarbonitrile (3.0 g, 5.76 mmol) in dichloromethane (10 mL) was added trifluoroacetic acid (15 ml, 197 mmol) dropwise at 10° C. and the mixture was stirred at 10° C. for 4 h. The reaction mixture was diluted with water (100 mL) and adjusted pH to 7 with saturated sodium bicarbonate solution at 0-10° C. The mixture was extracted with ethyl acetate (50 mL×2). The combined organic layers were washed with brine (50 mL×2), dried over sodium sulfate, filtered and concentrated under reduced pressure. The residue was triturated with ethyl acetate (4 mL) and filtered. The filter cake was dried under reduced pressure to give the title compound (907 mg, 70% yield) as a white solid. MS (ESI) m/z: 221.1 [M+H]⁺

### EXAMPLES

### Example 1: N-[5-(2,2-difluoroethoxy)-4,6-dimethoxy-pyrimidin-2-yl]-5-phenyl-1H-pyrrole-3-sulfonamide

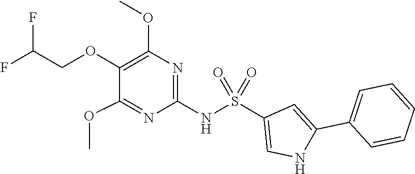 [SMILES: COc1nc(NS(=O)(=O)c2c[nH]c(-c3ccccc3)c2)nc(OC)c1OCC(F)F]

A solution of [5-(2,2-difluoroethoxy)-4,6-dimethoxy-pyrimidin-2-yl]amine (30 mg, 125.01 umol, intermediate B4) in acetonitrile (1.2 mL) was cooled to 0° C. 2 M sodium tert-butoxide solution in THF (93.75 uL, 187.51 umol) was added dropwise. The reaction mixture was stirred at 0° C. for 15 min. A solution of 5-phenyl-1H-pyrrole-3-sulfonyl chloride (36.93 mg, 137.51 umol, intermediate A2) in acetonitrile (600 uL) was added dropwise. The reaction mixture was allowed to warm to room temperature, stirred for 1 hour, quenched with a saturated ammonium chloride solution and extracted twice with ethylacetate. The organic layers were dried over sodium sulfate, filtered and concentrated in vacuo. The residue was purified by flash chromatography over silica gel using a gradient ethylacetate/heptane 0-60%, followed by another flash chromatography on a C18 RediSepRf Gold column using a gradient acetonitrile/water 10-70% to provide the title compound as a white solid (7.9 mg, 14%). MS (ESI) m/z: 441.2 [M+H]+

### Example 2: N-[5-(difluoromethoxy)-4,6-dimethoxy-pyrimidin-2-yl]-5-phenyl-1H-pyrrole-3-sulfonamide

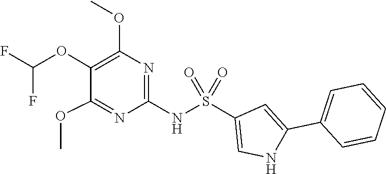 [SMILES: COc1nc(NS(=O)(=O)c2c[nH]c(-c3ccccc3)c2)nc(OC)c1OC(F)F]

The title compound was prepared following same procedure as for example 1 from intermediate A2 and intermediate B1 (white solid, 7.3% yield). MS (ESI) m/z: 427.2 [M+H]+

### Example 3: N-[5-(2-fluoroethoxy)-4,6-dimethoxy-pyrimidin-2-yl]-5-phenyl-1H-pyrrole-3-sulfonamide

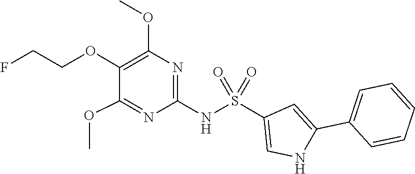 [SMILES: COc1nc(NS(=O)(=O)c2c[nH]c(-c3ccccc3)c2)nc(OC)c1OCCF]

To a mixture of [5-(2-fluoroethoxy)-4,6-dimethoxy-pyrimidin-2-yl]amine (55 mg, 235.5 umol, intermediate B3) and 5-phenyl-1H-pyrrole-3-sulfonyl chloride (73.99 mg, 306.15 umol, intermediate A2) was added pyridine (1.1 mL). The reaction mixture was stirred at room temperature for 16 hours, poured into brine and extracted twice with ethylacetate. The organic layers were dried over sodium sulfate, filtered and concentrated in vacuo. The residue was purified by flash chromatography over silica gel using a gradient ethylacetate/heptane 0-60% to provide the title compound as a off-white solid (26.7 mg, 27%). MS (ESI) m/z: 423.1 [M+H]+

### Example 4: N-[5-(2,2-difluoroethoxy)-4,6-dimethoxy-pyrimidin-2-yl]-5-thiazol-2-yl-1H-pyrrole-3-sulfonamide

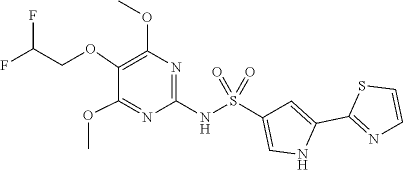 [SMILES: COc1nc(NS(=O)(=O)c2c[nH]c(-c3nccs3)c2)nc(OC)c1OCC(F)F]

The title compound was prepared following same procedure as for example 3 from intermediate A1 and intermediate B4 (off-white solid, 15.5% yield). MS (ESI) m/z: 448.1 [M+H]+

### Example 5: N-[5-(difluoromethoxy)-4,6-dimethoxy-pyrimidin-2-yl]-5-(3-fluoro-2-pyridyl)-1H-pyrrole-3-sulfonamide

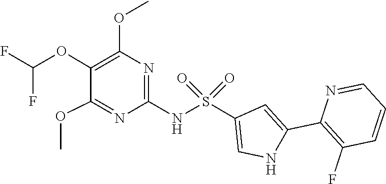 [SMILES: COc1nc(NS(=O)(=O)c2c[nH]c(-c3ncccc3F)c2)nc(OC)c1OC(F)F]

To a solution of [5-(difluoromethoxy)-4,6-dimethoxy-pyrimidin-2-yl]amine (50 mg, 226.08 umol, intermediate B1) and 5-(3-fluoro-2-pyridyl)-1H-pyrrole-3-sulfonyl chloride (71.43 mg, 271.3 umol, intermediate A3 in acetonitrile (5 mL) was added diisopropylethylamine (87.66 mg, 118.45 uL, 678.24 umol). The solution was stirred at 60° C. overnight. The solvent was evaporated in vacuo, the residue was dissolved in ethyl acetate/water and extracted with ethyl acetate. The combined organic layers were washed with brine, dried over sodium sulfate, filtered and concentrated in vacuo. The residue was purified by flash chromatography over silica gel using a gradient ethylacetate/heptane 0-50% to provide the title compound as a white solid (13 mg, 13%). MS (ESI) m/z: 446.1 [M+H]+

### Example 6: N-[5-(2,2-difluoroethyl)-4,6-dimethoxy-pyrimidin-2-yl]-5-(3-fluoro-2-pyridyl)-1H-pyrrole-3-sulfonamide

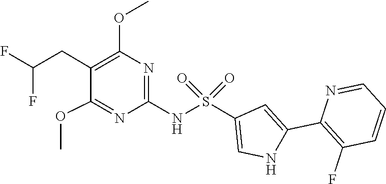 [SMILES: COc1nc(NS(=O)(=O)c2c[nH]c(-c3ncccc3F)c2)nc(OC)c1CC(F)F]

The title compound was prepared following same procedure as for example 5 from intermediate A3 and intermediate B2 (white solid, 18.2% yield). MS (ESI) m/z: 444.1 [M+H]+

### Example 7: N-[5-(2-fluoroethoxy)-4,6-dimethoxy-pyrimidin-2-yl]-5-(3-fluoro-2-pyridyl)-1H-pyrrole-3-sulfonamide

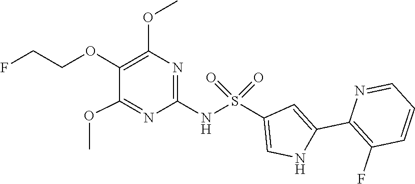 [SMILES: COc1nc(NS(=O)(=O)c2c[nH]c(-c3ncccc3F)c2)nc(OC)c1OCCF]

The title compound was prepared following same procedure as for example 5 from intermediate A3 and intermediate B3 (white solid, 31.2% yield). MS (ESI) m/z: 442.1 [M+H]+

### Example 8: N-[5-(2,2-difluoroethoxy)-4,6-dimethoxy-pyrimidin-2-yl]-5-(3-fluoro-2-pyridyl)-1H-pyrrole-3-sulfonamide

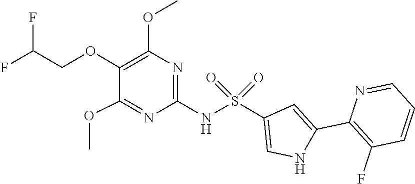 [SMILES: COc1nc(NS(=O)(=O)c2c[nH]c(-c3ncccc3F)c2)nc(OC)c1OCC(F)F]

The title compound was prepared following same procedure as for example 5 from intermediate A3 and intermediate B4 (white solid, 42% yield). MS (ESI) m/z: 460.1 [M+H]+

### Example 9: N-[5-(2,2-difluoroethoxy)-4-methoxy-pyrimidin-2-yl]-5-thiazol-2-yl-1H-pyrrole-3-sulfonamide

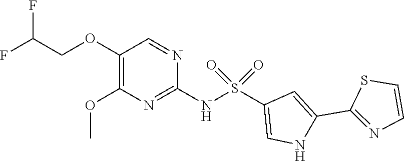 [SMILES: COc1nc(NS(=O)(=O)c2c[nH]c(-c3nccs3)c2)ncc1OCC(F)F]

To 5-thiazol-2-yl-1H-pyrrole-3-sulfonyl chloride (89 mg, 0.351 mmol, Intermediate A1) and [5-(2,2-difluoroethoxy)-4-methoxy-pyrimidin-2-yl]amine (60 mg, 0.292 mmol, Intermediate B5) was added pyridine (2.68 g, 2.7 mL) at room temperature. The resulting solution was stirred at 60° C. for 20 h. The reaction mixture was diluted with ethyl acetate and water. The aqueous layer was extracted two times with ethyl acetate. The combined organic layers were washed with water, dried over sodium sulfate, filtered and then concentrated in vacuo. The residue was purified by chromatography (silica gel, ethyl acetate in heptane, 0 to 100%) to afford N-[5-(2,2-difluoroethoxy)-4-methoxy-pyrimidin-2-yl]-5-thiazol-2-yl-1H-pyrrole-3-sulfonamide (34 mg, 27% yield) as white solid. MS (ESI) m/z: 418.0 [M+H]⁺.

### Example 10: N-[5-(2-cyanoethyl)-4,6-dimethoxy-pyrimidin-2-yl]-5-thiazol-2-yl-1H-pyrrole-3-sulfonamide

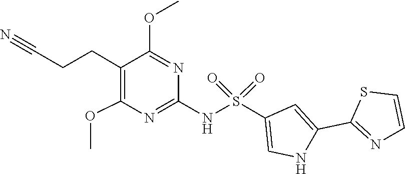 [SMILES: COc1nc(NS(=O)(=O)c2c[nH]c(-c3nccs3)c2)nc(OC)c1CCC#N]

The title compound was prepared in analogy to Example 9 from 3-(2-amino-4,6-dimethoxy-pyrimidin-5-yl)propionitrile (Intermediate B7) instead of Intermediate B5 as white solid. MS (ESI) m/z: 421.0 [M+H]⁺.

### Example 11: N-[5-(2,2-difluoroethoxy)-4,6-dimethoxy-pyrimidin-2-yl]-5-isothiazol-3-yl-1H-pyrrole-3-sulfonamide

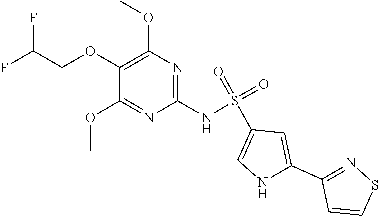 [SMILES: COc1nc(NS(=O)(=O)c2c[nH]c(-c3ccsn3)c2)nc(OC)c1OCC(F)F]

To a stirred solution of [5-(2,2-difluoroethoxy)-4,6-dimethoxy-pyrimidin-2-yl]amine (28 mg, 115 umol, Intermediate B4) and N-ethyldiisopropylamine (30 mg, 39 uL, 0.231 mmol) in 1,2-dichloroethane (0.3 mL) was added 5-isothiazol-3-yl-1H-pyrrole-3-sulfonyl chloride (43 mg, 0.139 mmol, Intermediate A11) in 1,2-dichloroethane (0.8 mL) dropwise at room temperature.

The reaction was stirred at 70° C. overnight. The reaction mixture was cooled to room temperature, poured into brine, and extracted twice with ethyl acetate. The organic layers were washed with water and brine, dried with Na2SO4, filtered and concentrated in vacuo. The residue was purified by flash column chromatography (12 g, 0% to 60% ethyl acetate in heptane) to provide N-[5-(2,2-difluoroethoxy)-4,6-dimethoxy-pyrimidin-2-yl]-5-isothiazol-3-yl-1H-pyrrole-3-sulfonamide (8 mg, 15% yield) as off-white solid. MS (ESI) m/z: 448.1 [M+H]⁺.

### Example 12: N-[5-(2,2-difluoroethyl)-4,6-dimethoxy-pyrimidin-2-yl]-5-phenyl-1H-pyrrole-3-sulfonamide

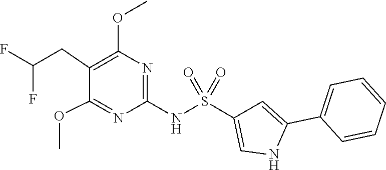 [SMILES: COc1nc(NS(=O)(=O)c2c[nH]c(-c3ccccc3)c2)nc(OC)c1CC(F)F]

The title compound was prepared in analogy to Example 11 from 5-(2,2-difluoroethyl)-4,6-dimethoxy-pyrimidin-2-amine (Intermediate B2) instead of Intermediate B4 and 5-phenyl-1H-pyrrole-3-sulfonyl chloride (Intermediate A2) instead of Intermediate A11 as off-white solid. MS (ESI) m/z: 425.1 [M+H]⁺.

The following Examples 13-19 were prepared in analogy to Example 5 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 13 | 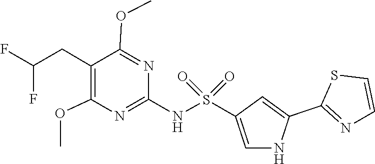 [SMILES: COc1nc(NS(=O)(=O)c2c[nH]c(-c3nccs3)c2)nc(OC)c1CC(F)F] | N-[5-(2,2-difluoroethyl)- 4,6-dimethoxy-pyrimidin-2- yl]-5-thiazol-2-yl-1H- pyrrole-3-sulfonamide | A1 | B2 | 432.0 [M + H]⁺ |
| 14 | 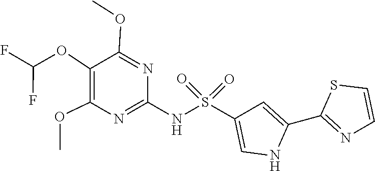 [SMILES: COc1nc(NS(=O)(=O)c2c[nH]c(-c3nccs3)c2)nc(OC)c1OC(F)F] | N-[5-(difluoromethoxy)- 4,6-dimethoxy-pyrimidin-2- yl]-5-thiazol-2-yl-1H- pyrrole-3-sulfonamide | A1 | B1 | 434.0 [M + H]⁺ |
| 15 | 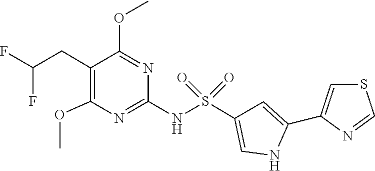 [SMILES: COc1nc(NS(=O)(=O)c2c[nH]c(-c3cscn3)c2)nc(OC)c1CC(F)F] | N-[5-(2,2-difluoroethyl)- 4,6-dimethoxy-pyrimidin-2- yl]-5-thiazol-4-yl-1H- pyrrole-3-sulfonamide | A9 | B2 | 432.0 [M + H]⁺ |
| 16 | 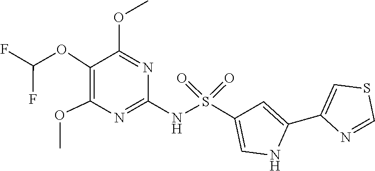 [SMILES: COc1nc(NS(=O)(=O)c2c[nH]c(-c3cscn3)c2)nc(OC)c1OC(F)F] | N-[5-(difluoromethoxy)- 4,6-dimethoxy-pyrimidin-2- yl]-5-thiazol-4-yl-1H- pyrrole-3-sulfonamide | A9 | B1 | 434.0 [M + H]⁺ |
| 17 | 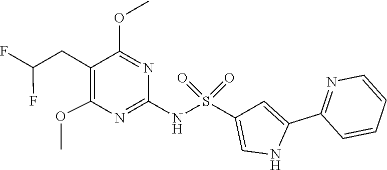 [SMILES: COc1nc(NS(=O)(=O)c2c[nH]c(-c3ccccn3)c2)nc(OC)c1CC(F)F] | N-[5-(2,2-difluoroethyl)- 4,6-dimethoxy-pyrimidin-2- yl]-5-(2-pyridyl)-1H- pyrrole-3-sulfonamide | A7 | B2 | 426.1 [M + H]⁺ |
| 18 | 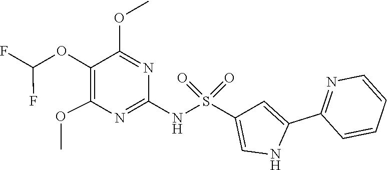 [SMILES: COc1nc(NS(=O)(=O)c2c[nH]c(-c3ccccn3)c2)nc(OC)c1OC(F)F] | N-[5-(difluoromethoxy)- 4,6-dimethoxy-pyrimidin-2- yl]-5-(2-pyridyl)-1H- pyrrole-3-sulfonamide | A7 | B1 | 428.1 [M + H]⁺ |
| 19 | 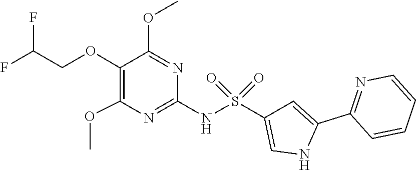 [SMILES: COc1nc(NS(=O)(=O)c2c[nH]c(-c3ccccn3)c2)nc(OC)c1OCC(F)F] | N-[5-(2,2-difluoroethoxy)- 4,6-dimethoxy-pyrimidin-2- yl]-5-(2-pyridyl)-1H- pyrrole-3-sulfonamide | A7 | B4 | 442.1 [M + H]⁺ |

### Example 20: N-[5-(2,2-difluoroethyl)-4,6-dimethoxy-pyrimidin-2-yl]-5-(4-methylthiazol-2-yl)-1H-pyrrole-3-sulfonamide

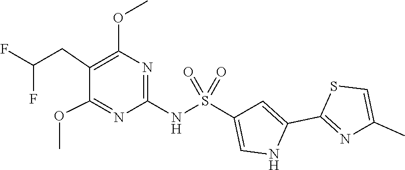 [SMILES: COc1nc(NS(=O)(=O)c2c[nH]c(-c3nc(C)cs3)c2)nc(OC)c1CC(F)F]

To a stirred solution of [5-(2,2-difluoroethyl)-4,6-dimethoxy-pyrimidin-2-yl]amine (110 mg, 0.472 mmol, intermediate B2) and N-ethyldiisopropylamine (187 mg, 247 uL, 1.42 mmol) in dichloromethane (2 mL) was added a solution of 5-(4-methylthiazol-2-yl)-1H-pyrrole-3-sulfonyl chloride (161 mg, 0.613 mmol, intermediate A8) in ethyl acetate (1 mL) dropwise at room temperature and the mixture was stirred at room temperature for 16 h. The reaction mixture was poured into brine and extracted twice with ethyl acetate. The organic layers were dried over sodium sulfate, filtered and concentrated in vacuo. The residue was purified by flash chromatography (silica gel, 0% to 70% ethyl acetate in heptane) to provide N-[5-(2,2-difluoroethyl)-4,6-dimethoxy-pyrimidin-2-yl]-5-(4-methylthiazol-2-yl)-1H-pyrrole-3-sulfonamide (35 mg, 17% yield) as off-white solid. MS (ESI) m/z: 446.3 [M+H]⁺.

### Example 21: 4-benzyl-N-[5-(2,2-difluoroethyl)-4,6-dimethoxy-pyrimidin-2-yl]-1H-pyrrole-3-sulfonamide

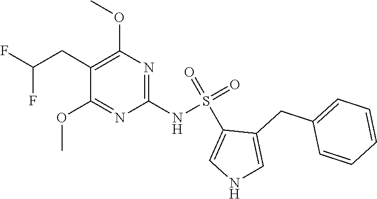 [SMILES: COc1nc(NS(=O)(=O)c2c[nH]cc2Cc2ccccc2)nc(OC)c1CC(F)F]

To a stirred suspension of [5-(2,2-difluoroethyl)-4,6-dimethoxy-pyrimidin-2-yl]amine (100 mg, 0.365 mmol, intermediate B2) and tripotassium phosphate (286 mg, 1.35 mmol) in acetonitrile (800 uL) was added 4-benzyl-1H-pyrrole-3-sulfonyl chloride (389 mg, 0.547 mmol, intermediate A5) in acetonitrile (2.2 mL) dropwise at room temperature. The reaction mixture was stirred for 3 days at 70° C. The reaction mixture was allowed to room temperature, poured into brine and extracted with ethyl acetate/methanol. The organic layers were dried over Na2SO4, filtered and concentrated in vacuo. The residue was purified by flash column chromatography (silica, 0% to 60% ethyl acetate in heptane), followed by reversed-phase flash column chromatography (column YMC-Triart C18, 12 nm, 5 μm, 100×30 mm, acetonitrile/water+0.1% HCOOH) to provide 4-benzyl-N-[5-(2,2-difluoroethyl)-4,6-dimethoxy-pyrimidin-2-yl]-1H-pyrrole-3-sulfonamide (5 mg, 3% yield) as white powder. MS (ESI) m/z: 439.1 [M+H]⁺.

### Example 22: 4-benzyl-N-[5-(difluoromethoxy)-4,6-dimethoxy-pyrimidin-2-yl]-1H-pyrrole-3-sulfonamide

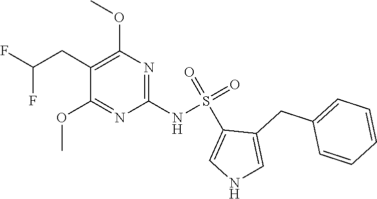 [SMILES: COc1nc(NS(=O)(=O)c2c[nH]cc2Cc2ccccc2)nc(OC)c1CC(F)F]

The title compound was prepared in analogy to Example 21 from 5-(difluoromethoxy)-4,6-dimethoxy-pyrimidin-2-amine (Intermediate B1) instead of Intermediate B2 as white solid. MS (ESI) m/z: 441.1 [M+H]⁺.

### Example 23: N-[5-(2-cyanoethyl)-4,6-dimethoxy-pyrimidin-2-yl]-5-(3-fluoro-2-pyridyl)-1H-pyrrole-3-sulfonamide

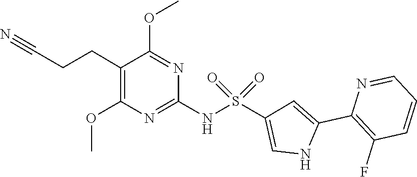 [SMILES: COc1nc(NS(=O)(=O)c2c[nH]c(-c3ncccc3F)c2)nc(OC)c1CCC#N]

A mixture of 3-(2-amino-4,6-dimethoxy-pyrimidin-5-yl)propionitrile (50 mg, 0.240 mmol, intermediate B7) and 5-(3-fluoro-2-pyridyl)-1H-pyrrole-3-sulfonyl chloride (76 mg, 0.288 mmol, intermediate A3) in acetonitrile (1 mL) and pyridine (1 mL) was stirred at 60° C. overnight. The reaction mixture was concentrated in vacuo, the residue was dissolved in ethyl acetate/water, poured into water and extracted with ethyl acetate twice. The combined organic layers were washed with brine, dried over sodium sulfate, filtered and concentrated in vacuo. The residue was purified by flash chromatography (silica gel, 0% to 80% ethyl acetate in heptane) to afford N-[5-(2-cyanoethyl)-4,6-dimethoxy-pyrimidin-2-yl]-5-(3-fluoro-2-pyridyl)-1H-pyrrole-3-sulfonamide (34 mg, 32% yield) as grey solid. MS (ESI) m/z: 433.1 [M+H].

The following Examples 24-28 were prepared in analogy to Example 23 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 24 | 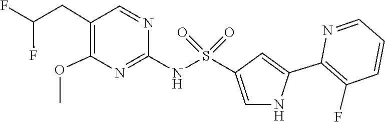 [SMILES: COc1nc(NS(=O)(=O)c2c[nH]c(-c3ncccc3F)c2)ncc1CC(F)F] | N-[5-(2,2-difluoroethyl)-4- methoxy-pyrimidin-2-yl]-5- (3-fluoro-2-pyridyl)-1H- pyrrole-3-sulfonamide | A3 | B6 | 414.1 [M + H]⁺ |
| 25 | 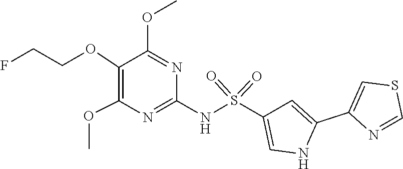 [SMILES: COc1nc(NS(=O)(=O)c2c[nH]c(-c3cscn3)c2)nc(OC)c1OCCF] | N-[5-(2-fluoroethoxy)-4,6- dimethoxy-pyrimidin-2-yl]- 5-thiazol-4-yl-1H-pyrrole-3- sulfonamide | A9 | B3 | 430.0 [M + H]⁺ |
| 26 | 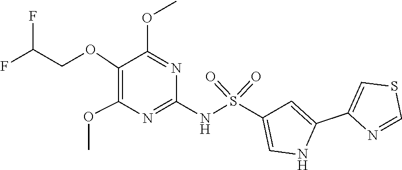 [SMILES: COc1nc(NS(=O)(=O)c2c[nH]c(-c3cscn3)c2)nc(OC)c1OCC(F)F] | N-[5-(2,2-difluoroethoxy)- 4,6-dimethoxy-pyrimidin-2- yl]-5-thiazol-4-yl-1H- pyrrole-3-sulfonamide | A9 | B4 | 448.0 [M + H]⁺ |
| 27 | 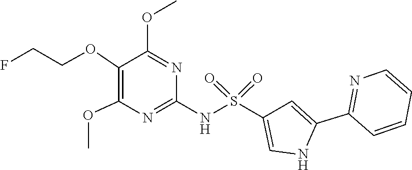 [SMILES: COc1nc(NS(=O)(=O)c2c[nH]c(-c3ccccn3)c2)nc(OC)c1OCCF] | N-[5-(2-fluoroethoxy)-4,6- dimethoxy-pyrimidin-2-yl]- 5-(2-pyridyl)-1H-pyrrole-3- sulfonamide | A7 | B3 | 424.1 [M + H]⁺ |
| 28 | 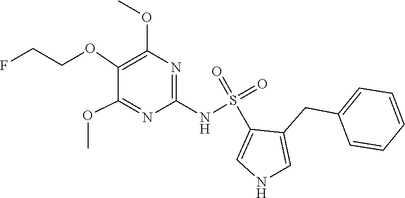 [SMILES: COc1nc(NS(=O)(=O)c2c[nH]cc2Cc2ccccc2)nc(OC)c1OCCF] | 4-benzyl-N-[5-(2- fluoroethoxy)-4,6- dimethoxy-pyrimidin-2-yl]- 1H-pyrrole-3-sulfonamide | A5 | B3 | 437.1 [M + H]⁺ |

### Example 29: 4-[dideuterio-(3-fluorophenyl)methyl]-N-[5-(2,2-difluoroethoxy)-4,6-dimethoxy-pyrimidin-2-yl]-1H-pyrrole-3-sulfonamide

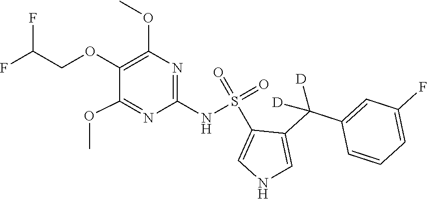 [SMILES: [2H]C([2H])(c1cccc(F)c1)c1c[nH]cc1S(=O)(=O)Nc1nc(OC)c(OCC(F)F)c(OC)n1]

To a stirred solution of [5-(2,2-difluoroethoxy)-4,6-dimethoxy-pyrimidin-2-yl]amine (55 mg, 0.227 mmol, intermediate B4) in pyridine (0.5 mL) was added 4-[dideuterio-(3-fluorophenyl)methyl]-1H-pyrrole-3-sulfonyl chloride (179 mg, 0.272 mmol, intermediate A13) in acetonitrile (1.5 mL) dropwise at room temperature and the mixture was stirred for 24 h at room temperature. The reaction mixture was poured into brine and extracted three times with ethyl acetate. The organic layers were dried with Na2SO4, filtered and concentrated in vacuo. The residue was purified by flash column chromatography (silica gel, 000 to 55% ethyl acetate in heptane) to provide 4-[dideuterio-(3-fluorophenyl)methyl]-N-[5-(2,2-difluoroethoxy)-4,6-dimethoxy-pyrimidin-2-yl]-1H-pyrrole-3-sulfonamide (39 mg, 35% yield) as light brown solid. MS (ESI) m/z: 475.1 [M+H]⁺.

### Example 30: 4-benzyl-N-[5-(2,2-difluoroethoxy)-4,6-dimethoxy-pyrimidin-2-yl]-1H-pyrrole-3-sulfonamide

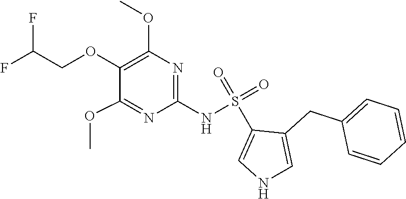 [SMILES: COc1nc(NS(=O)(=O)c2c[nH]cc2Cc2ccccc2)nc(OC)c1OCC(F)F]

The title compound was prepared in analogy to Example 29 from 4-benzyl-TH-pyrrole-3-sulfonyl chloride (Intermediate A5) instead of Intermediate A13 as white powder. MS (ESI) m/z: 455.1 [M+H]⁺.

The following Examples 31-33 were prepared in analogy to Example 9 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 31 | 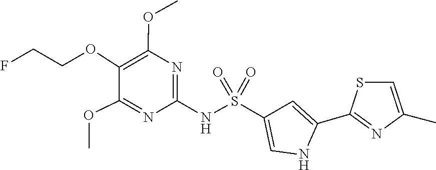 [SMILES: COc1nc(NS(=O)(=O)c2c[nH]c(-c3nc(C)cs3)c2)nc(OC)c1OCCF] | N-[5-(2-fluoroethoxy)-4,6- dimethoxy-pyrimidin-2-yl]- 5-(4-methylthiazol-2-yl)- 1H-pyrrole-3-sulfonamide | A8 | B3 | 444.1 [M + H]⁺ |
| 32 | 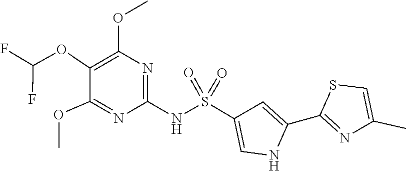 [SMILES: COc1nc(NS(=O)(=O)c2c[nH]c(-c3nc(C)cs3)c2)nc(OC)c1OC(F)F] | N-[5-(difluoromethoxy)- 4,6-dimethoxy-pyrimidin-2- yl]-5-(4-methylthiazol-2- yl)-1H-pyrrole-3- sulfonamide | A8 | B1 | 448.0 [M + H]⁺ |
| 33 | 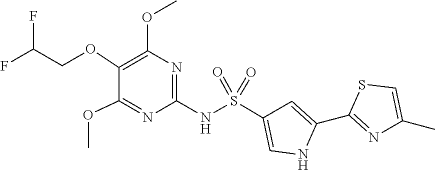 [SMILES: COc1nc(NS(=O)(=O)c2c[nH]c(-c3nc(C)cs3)c2)nc(OC)c1OCC(F)F] | N-[5-(2,2-difluoroethoxy)- 4,6-dimethoxy-pyrimidin-2- yl]-5-(4-methylthiazol-2- yl)-1H-pyrrole-3- sulfonamide | A8 | B4 | 462.3 [M + H]⁺ |

The following Examples 34-41 were prepared in analogy to Example 3 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 34 | 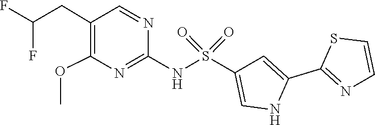 [SMILES: COc1nc(NS(=O)(=O)c2c[nH]c(-c3nccs3)c2)ncc1CC(F)F] | N-[5-(2,2-difluoroethyl)-4- methoxy-pyrimidin-2-yl]-5- thiazol-2-yl-1H-pyrrole-3- sulfonamide | A1 | B6 | 402.0 [M + H]⁺ |
| 35 | 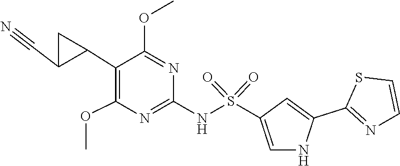 [SMILES: COc1nc(NS(=O)(=O)c2c[nH]c(-c3nccs3)c2)nc(OC)c1C1CC1C#N] | N-[5-(2-cyanocyclopropyl)- 4,6-dimethoxy-pyrimidin-2- yl]-5-thiazol-2-yl-1H- pyrrole-3-sulfonamide | A1 | B8 | 433.1 [M + H]⁺ |
| 36 | 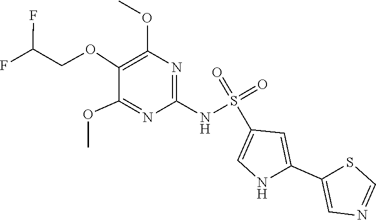 [SMILES: COc1nc(NS(=O)(=O)c2c[nH]c(-c3cncs3)c2)nc(OC)c1OCC(F)F] | N-[5-(2,2-difluoroethoxy)- 4,6-dimethoxy-pyrimidin-2- yl]-5-thiazol-5-yl-1H- pyrrole-3-sulfonamide | A10 | B4 | 448.1 [M + H]⁺ |
| 37 | 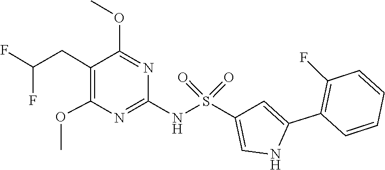 [SMILES: COc1nc(NS(=O)(=O)c2c[nH]c(-c3ccccc3F)c2)nc(OC)c1CC(F)F] | N-[5-(2,2-difluoroethyl)- 4,6-dimethoxy-pyrimidin-2- yl]-5-(2-fluorophenyl)-1H- pyrrole-3-sulfonamide | A6 | B2 | 443.1 [M + H]⁺ |
| 38 | 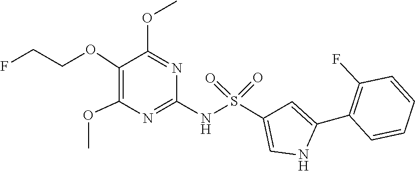 | N-[5-(2-fluoroethoxy)-4,6- dimethoxy-pyrimidin-2-yl]- 5-(2-fluorophenyl)-1H- pyrrole-3-sulfonamide | A6 | B3 | 444.1 [M + H]⁺ |
| 39 | 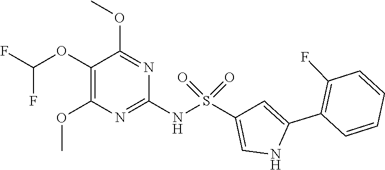 [SMILES: COc1nc(NS(=O)(=O)c2c[nH]c(-c3ccccc3F)c2)nc(OC)c1OC(F)F] | N-[5-(difluoromethoxy)- 4,6-dimethoxy-pyrimidin-2- yl]-5-(2-fluorophenyl)-1H- pyrrole-3-sulfonamide | A6 | B1 | 445.1 [M + H]⁺ |
| 40 | 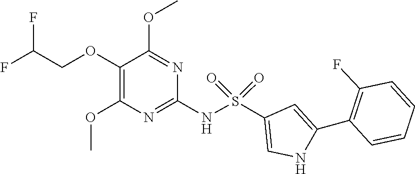 [SMILES: COc1nc(NS(=O)(=O)c2c[nH]c(-c3ccccc3F)c2)nc(OC)c1OCC(F)F] | N-[5-(2,2-difluoroethoxy)- 4,6-dimethoxy-pyrimidin-2- yl]-5-(2-fluorophenyl)-1H- pyrrole-3-sulfonamide | A6 | B4 | 459.1 [M + H]⁺ |
| 41 | 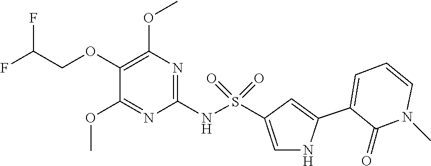 [SMILES: COc1nc(NS(=O)(=O)c2c[nH]c(-c3cccn(C)c3=O)c2)nc(OC)c1OCC(F)F] | N-[5-(2,2-difluoroethoxy)- 4,6-dimethoxy-pyrimidin-2- yl]-5-(2-keto-1-methyl-3- pyridyl)-1H-pyrrole-3- sulfonamide | A12 | B4 | 472.1 [M + H]⁺ |

### Example 42: N-[5-(2,2-difluoroethoxy)-4,6-dimethoxy-pyrimidin-2-yl]-5-(2-methylthiazol-5-yl)-1H-pyrrole-3-sulfonamide

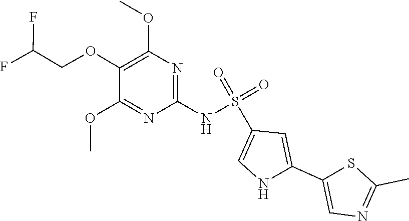 [SMILES: COc1nc(NS(=O)(=O)c2c[nH]c(-c3cnc(C)s3)c2)nc(OC)c1OCC(F)F]

### Step 1: N-[5-(2,2-difluoroethoxy)-4,6-dimethoxy-pyrimidin-2-yl]-1-(p-tolylsulfonyl)pyrrole-3-sulfonamide

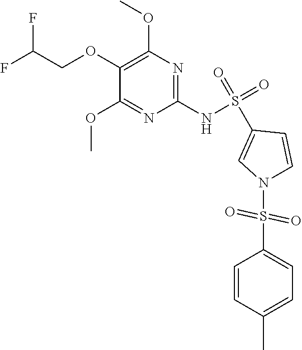 [SMILES: COc1nc(NS(=O)(=O)c2ccn(S(=O)(=O)c3ccc(C)cc3)c2)nc(OC)c1OCC(F)F]

To a mixture of 5-(2,2-difluoroethoxy)-4,6-dimethoxy-pyrimidin-2-amine (3.2 g, 13.6 mmol, intermediate B4) in pyridine (60 mL) was added 1-(p-tolylsulfonyl)pyrrole-3-sulfonyl chloride (8.7 g, 27.21 mmol, CAS 881406-27-5, see WO2022254027) at 0° C., and the mixture was stirred at 70° C. for 12 h under nitrogen atmosphere. The reaction mixture was diluted with water (100 mL) and extracted with ethyl acetate (100 mL×3). The combined organic layers were washed with saturated sodium chloride solution (50 mL), dried over sodium sulfate, filtered and concentrated under reduced pressure. The residue was triturated with methanol (50 mL) at 20° C. for 30 min to give 3.8 g of a first batch of product. The mother liquor was concentrated under reduced pressure and purified by flash silica gel chromatography (silica gel, 0-45% ethyl acetate in petroleum ether) to give a crude product which was further purified by preparative HPLC (Phenomenex Luna C8, 250×50 mm×10 um, water (0.1% formic acid)/acetonitrile) to a second batch of product (2.0 g) which was combined with the first batch to yield N-[5-(2,2-difluoroethoxy)-4,6-dimethoxy-pyrimidin-2-yl]-1-(p-tolylsulfonyl)pyrrole-3-sulfonamide (5.8 g, 76% yield) as an off-white solid. MS (ESI) m/z: 519.0 [M+H]⁺.

### Step 2: N-[5-(2,2-difluoroethoxy)-4,6-dimethoxy-pyrimidin-2-yl]-1H-pyrrole-3-sulfonamide

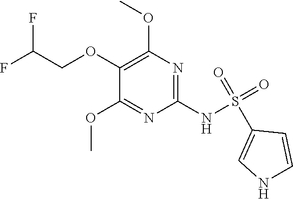 [SMILES: COc1nc(NS(=O)(=O)c2cc[nH]c2)nc(OC)c1OCC(F)F]

To a mixture of N-[5-(2,2-difluoroethoxy)-4,6-dimethoxy-pyrimidin-2-yl]-1-(p-tolylsulfonyl)pyrrole-3-sulfonamide (3.6 g, 6.94 mmol) in methanol (36 mL) was added sodium hydroxide (4 M in water, 8.7 mL, 34.7 mmol) at 20° C. under nitrogen and the mixture was stirred at 80° C. for 2 h. The reaction mixture was quenched by addition of 1M hydrochloric acid at 20° C. until the pH=7, then concentrated under reduced pressure. The residue was purified by preparative HPLC (Phenomenex Luna C8, 250×50 mm×10 um, water (0.1% formic acid)/acetonitrile) to give N-[5-(2,2-difluoroethoxy)-4,6-dimethoxy-pyrimidin-2-yl]-1H-pyrrole-3-sulfonamide (2.15 g, 84% yield) as a light yellow solid. MS (ESI) m/z: 365.0 [M+H]⁺.

### Step 3: 5-bromo-N-[5-(2,2-difluoroethoxy)-4,6-dimethoxy-pyrimidin-2-yl]-1H-pyrrole-3-sulfonamide

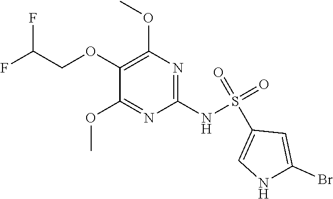 [SMILES: COc1nc(NS(=O)(=O)c2c[nH]c(Br)c2)nc(OC)c1OCC(F)F]

To a mixture of N-[5-(2,2-difluoroethoxy)-4,6-dimethoxy-pyrimidin-2-yl]-1H-pyrrole-3-sulfonamide (1.03 g, 2.83 mmol) in tetrahydrofuran (20 mL) was added N-bromosuccinimide (503 mg, 2.83 mmol) at −78° C., and the reaction was stirred at −78° C. for 2 h under nitrogen. The reaction mixture was diluted with water (50 mL) and extracted with ethyl acetate (100 mL×3). The combined organic layers were washed with saturated sodium chloride solution (50 mL), dried over sodium sulfate and concentrated under reduced pressure. The residue was purified by preparative HPLC (Phenomenex Luna C8, 250×50 mm×10 um, water (0.1% formic acid)/acetonitrile) and lyophilized to give 5-bromo-N-[5-(2,2-difluoroethoxy)-4,6-dimethoxy-pyrimidin-2-yl]-1H-pyrrole-3-sulfonamide (620 mg, 44% yield) as a light yellow solid. MS (ESI) m/z: 443.0 [M+H]⁺.

### Step 4: N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide

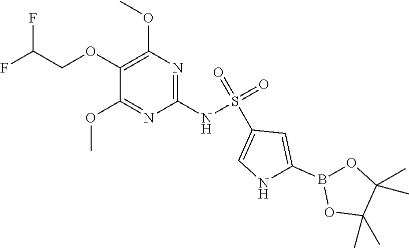 [SMILES: COc1nc(NS(=O)(=O)c2c[nH]c(B3OC(C)(C)C(C)(C)O3)c2)nc(OC)c1OCC(F)F]

To a mixture of 5-bromo-N-[5-(2,2-difluoroethoxy)-4,6-dimethoxy-pyrimidin-2-yl]-1H-pyrrole-3-sulfonamide (1.64 g, 3.7 mmol), bis(pinacolato)diboron (1.41 g, 5.55 mmol) and potassium acetate (726 mg, 7.4 mmol) in 1,4-dioxane (30 mL) was added [1,1′-bis(diphenylphosphino)ferrocene]palladium(II) chloride (271 mg, 0.37 mmol, 0.1 eq) at 20° C. under nitrogen, then the mixture was stirred at 100° C. for 2 h. The reaction mixture was diluted with water (10 mL) and extracted with ethyl acetate (10 mL×3). The combined organic layers were washed with saturated sodium chloride solution, dried over sodium sulfate and concentrated under reduced pressure. The residue was purified by flash silica gel chromatography (0-60% ethyl acetate in petroleum ether) to give N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide (870 mg, 46% yield) as a light yellow solid. MS (ESI) m/z: 491.1 [M+H]⁺.

### Step 5: N-[5-(2,2-difluoroethoxy)-4,6-dimethoxy-pyrimidin-2-yl]-5-(2-methylthiazol-5-yl)-1H-pyrrole-3-sulfonamide

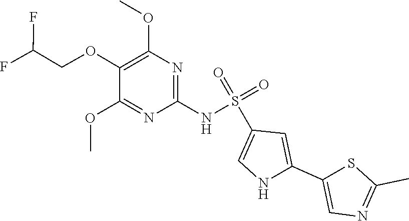 [SMILES: COc1nc(NS(=O)(=O)c2c[nH]c(-c3cnc(C)s3)c2)nc(OC)c1OCC(F)F]

To a mixture of N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide (50 mg, 0.1 mmol), 5-bromo-2-methyl-thiazole (18 mg, 0.1 mmol) and tripotassium phosphate (65 mg, 0.31 mmol) in n-butanol (2 mL) was added XPhos Pd G4 (8.8 mg, 0.01 mmol, 0.1 eq, CAS 1599466-81-5) at 20° C. under nitrogen, then the reaction was stirred at 80° C. for 2 h under nitrogen. The reaction mixture was filtered and purified by preparative HPLC (column Phenomenex Luna C18 150×25 mm×10 um, water+0.1% formic acid/acetonitrile) to give N-[5-(2,2-difluoroethoxy)-4,6-dimethoxy-pyrimidin-2-yl]-5-(2-methylthiazol-5-yl)-1H-pyrrole-3-sulfonamide (16 mg, 32% yield) as a white solid. MS (ESI) m/z: 462.0 [M+H].

### Example 43: N-[5-(2,2-difluoroethoxy)-4,6-dimethoxy-pyrimidin-2-yl]-5-pyrimidin-2-yl-1H-pyrrole-3-sulfonamide

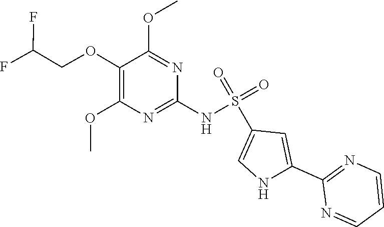 [SMILES: COc1nc(NS(=O)(=O)c2c[nH]c(-c3ncccn3)c2)nc(OC)c1OCC(F)F]

To a mixture of N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide (80 mg, 0.16 mmol, preparation see Example 42), 2-bromopyrimidine (26 mg, 0.16 mmol) and tripotassium phosphate (104 mg, 0.49 mmol) in n-butanol (0.2 mL) was added [(di(1-adamantyl)-butylphosphine)-2-(2′-amino-1,1′-biphenyl)]palladium(II) methanesulfonate, (12 mg, 0.02 mmol, 0.1 eq, CAS 1651823-59-4) at 20° C. under nitrogen and the reaction was stirred at 100° C. for 12 h. The reaction mixture was filtered and concentrated under reduced pressure. The residue was purified by preparative HPLC (Waters Xbridge 150×25 mm×5 um, water (with ammonia hydroxide)/acetonitrile) to give a crude product (30 mg) which was further purified by preparative TLC (silica gel, SiO2, petroleum ether/ethyl acetate=1:2) to yield N-[5-(2,2-difluoroethoxy)-4,6-dimethoxy-pyrimidin-2-yl]-5-pyrimidin-2-yl-1H-pyrrole-3-sulfonamide (6 mg, 8% yield) as a light yellow solid. MS (ESI) m/z: 443.0 [M+H].

### Example 44: N-[5-(2,2-difluoroethoxy)-4,6-dimethoxy-pyrimidin-2-yl]-5-(5-methylthiazol-2-yl)-1H-pyrrole-3-sulfonamide

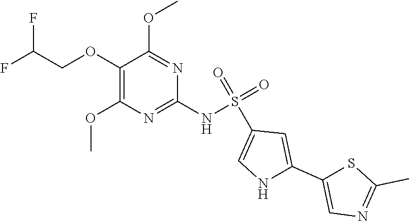 [SMILES: COc1nc(NS(=O)(=O)c2c[nH]c(-c3cnc(C)s3)c2)nc(OC)c1OCC(F)F]

To a mixture of N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide (5.0 mg, 0.01 mmol, preparation see Example 42), 2-bromo-5-methylthiazole (1.8 mg, 0.01 mmol), potassium carbonate (4.23 mg, 0.03 mmol) and lithium chloride (1.3 mg, 0.03 mmol) in 1,4-dioxane (0.2 mL) and water (0.01 mL) was added [1,1′-bis(diphenylphosphino)ferrocene]dichloropalladium(II), complex with dichloromethane (0.83 mg, 0.1 eq) at 20° C. under nitrogen, then the reaction was stirred at 90° C. for 3 h. The reaction mixture was filtered and purified by preparative HPLC (Phenomenex luna C18, 150×25 mm×10 um, water (0.1% formic acid)/acetonitrile) to give N-[5-(2,2-difluoroethoxy)-4,6-dimethoxy-pyrimidin-2-yl]-5-(5-methylthiazol-2-yl)-1H-pyrrole-3-sulfonamide (4.6 mg, 98% yield) as a white solid. MS (ESI) m/z: 462.1 [M+H]⁺.

### Example 45: N-[5-(2,2-difluoroethoxy)-4,6-dimethoxy-pyrimidin-2-yl]-5-pyridazin-3-yl-1H-pyrrole-3-sulfonamide

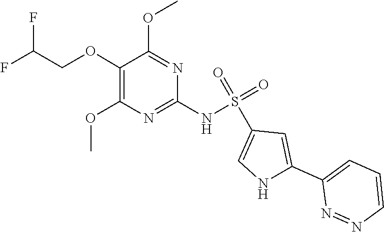 [SMILES: COc1nc(NS(=O)(=O)c2c[nH]c(-c3cccnn3)c2)nc(OC)c1OCC(F)F]

To a solution of N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide (80 mg, 0.16 mmol, preparation see Example 42), potassium carbonate (34 mg, 0.24 mmol), 3-bromopyridazine (26 mg, 0.16 mmol) in 1,4-dioxane (10 mL) and water (1.6 mL) was added tetrakis(triphenylphosphine)-palladium(0) (19 mg, 0.02 mmol, 0.1 eq) under nitrogen and the reaction mixture was stirred at 100° C. for 3 h. The reaction mixture was concentrated under reduced pressure and the residue purified by preparative HPLC (Phenomenex luna C18, 150×25 mm×10 um, water (0.1% formic acid)/acetonitrile) to give N-[5-(2,2-difluoroethoxy)-4,6-dimethoxy-pyrimidin-2-yl]-5-pyridazin-3-yl-1H-pyrrole-3-sulfonamide (11 mg, 15% yield) as a yellow solid. MS (ESI) m/z: 443.0 [M+H]⁺.

### Example 46: 5-(3-chloro-2-pyridyl)-N-[5-(2,2-difluoroethoxy)-4,6-dimethoxy-pyrimidin-2-yl]-1H-pyrrole-3-sulfonamide

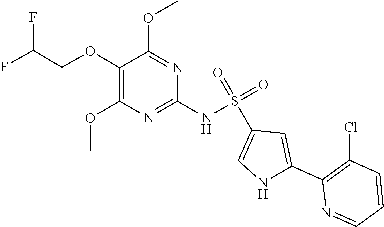 [SMILES: COc1nc(NS(=O)(=O)c2c[nH]c(-c3ncccc3Cl)c2)nc(OC)c1OCC(F)F]

The title compound was prepared in analogy to Example 45 from 2-bromo-3-chloropyridine instead of 3-bromopyridazine as brown gum. MS (ESI) m/z: 476.1 [M+H]⁺.

### Example 47: N-[5-(2,2-difluoroethoxy)-4,6-dimethoxy-pyrimidin-2-yl]-5-pyrimidin-4-yl-1H-pyrrole-3-sulfonamide

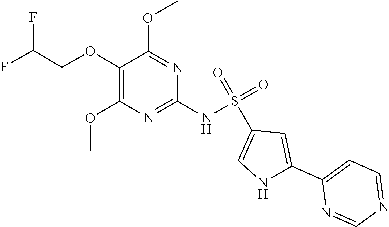 [SMILES: COc1nc(NS(=O)(=O)c2c[nH]c(-c3ccncn3)c2)nc(OC)c1OCC(F)F]

The title compound was prepared in analogy to Example 45 from 4-chloropyrimidine hydrochloride instead of 3-bromopyridazine as light yellow solid. MS (ESI) m/z: 443.0 [M+H]⁺.

### Example 48: N-[5-(2,2-difluoroethoxy)-4,6-dimethoxy-pyrimidin-2-yl]-5-pyrazin-2-yl-1H-pyrrole-3-sulfonamide

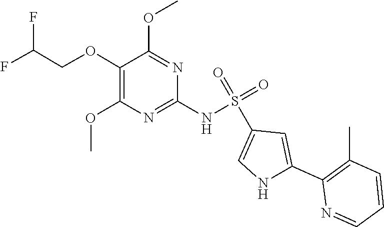 [SMILES: COc1nc(NS(=O)(=O)c2c[nH]c(-c3ncccc3C)c2)nc(OC)c1OCC(F)F]

To a mixture of N-[5-(2,2-difluoroethoxy)-4,6-dimethoxy-pyrimidin-2-yl]-5-(4,4,5,5-tetramethyl-1,3,2-dioxaborolan-2-yl)-1H-pyrrole-3-sulfonamide (80 mg, 0.16 mmol, preparation see Example 42), 2-bromopyrazine (26 mg, 0.16 mmol) and tripotassium phosphate (104 mg, 0.49 mmol) in n-butanol (0.3 mL) was added XPhos Pd G4 (14 mg, 0.02 mmol, 0.1 eq, CAS 1599466-81-5) at 20° C. under nitrogen and the mixture was stirred at 100° C. for 12 h. The reaction mixture was filtered and concentrated under reduced pressure. The residue was purified by preparative HPLC (Waters Xbridge 150×25 mm×5 um, water (with ammonium hydroxide)/acetonitrile) to give a crude product which was further purified by preparative TLC (silica gel, petroleum ether/ethyl acetate=1:2) to yield N-[5-(2,2-difluoroethoxy)-4,6-dimethoxy-pyrimidin-2-yl]-5-pyrazin-2-yl-1H-pyrrole-3-sulfonamide (15 mg, 20% yield) as a white solid. MS (ESI) m/z: 443.0 [M+H]⁺.

### Example 49: N-[5-(2,2-difluoroethoxy)-4,6-dimethoxy-pyrimidin-2-yl]-5-(3-methyl-2-pyridyl)-1H-pyrrole-3-sulfonamide

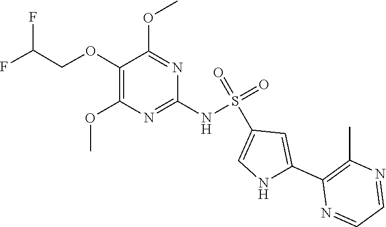 [SMILES: COc1nc(NS(=O)(=O)c2c[nH]c(-c3nccnc3C)c2)nc(OC)c1OCC(F)F]

The title compound was prepared in analogy to Example 48 from 2-bromo-3-methylpyridine instead of 2-bromopyrazine as yellow solid. MS (ESI) m/z: 456.1 [M+H]⁺.

### Example 50: 5-cyclobutyl-N-[5-(difluoromethoxy)-4,6-dimethoxy-pyrimidin-2-yl]-1H-pyrrole-3-sulfonamide

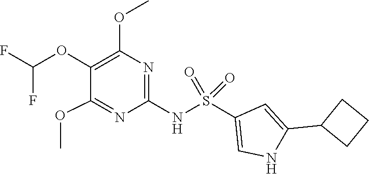 [SMILES: COc1nc(NS(=O)(=O)c2c[nH]c(C3CCC3)c2)nc(OC)c1OC(F)F]

### Step 1: 5-cyclobutyl-N-[5-(difluoromethoxy)-4,6-dimethoxy-pyrimidin-2-yl]-1-(p-tolylsulfonyl)pyrrole-3-sulfonamide

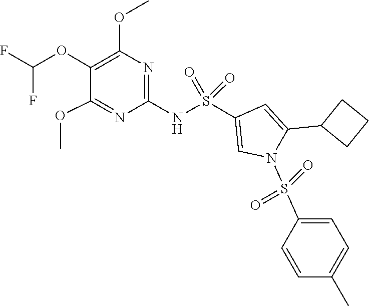 [SMILES: COc1nc(NS(=O)(=O)c2cc(C3CCC3)n(S(=O)(=O)c3ccc(C)cc3)c2)nc(OC)c1OC(F)F]

To a mixture of [5-(difluoromethoxy)-4,6-dimethoxy-pyrimidin-2-yl]amine (80 mg, 0.361 mmol, intermediate B1) and 5-cyclobutyl-1-tosyl-pyrrole-3-sulfonyl chloride (198 mg, 0.40 mmol2, intermediate A4) was added pyridine (2 mL). The mixture was stirred at room temperature for 2 h, then heated to 80° C. and stirred for 20 h. The reaction mixture was concentrated in vacuo and the residue was purified by flash chromatography (silica gel, 0% to 40% ethyl acetate in heptane) to provide 5-cyclobutyl-N-[5-(difluoromethoxy)-4,6-dimethoxy-pyrimidin-2-yl]-1-(p-tolylsulfonyl)pyrrole-3-sulfonamide (69 mg, 33% yield) as colorless gum. MS (ESI) m/z: 559.1 [M+H]⁺.

### Step 2: 5-cyclobutyl-N-[5-(difluoromethoxy)-4,6-dimethoxy-pyrimidin-2-yl]-1H-pyrrole-3-sulfonamide

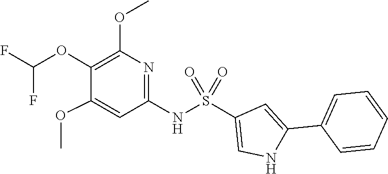 [SMILES: COc1cc(NS(=O)(=O)c2c[nH]c(-c3ccccc3)c2)nc(OC)c1OC(F)F]

To a stirred solution of 5-cyclobutyl-N-[5-(difluoromethoxy)-4,6-dimethoxy-pyrimidin-2-yl]-1-(p-tolylsulfonyl)pyrrole-3-sulfonamide (67 mg, 0.115 mmol) in methanol (1 mL) was added 5 M aqueous potassium hydroxide solution (185 uL, 0.92 mmol) and the mixture was stirred at 85° C. for 30 min. The reaction mixture was poured into water and extracted with ethyl acetate twice. The organic layers were dried over Na2SO4, filtered and concentrated in vacuo. The residue was purified by flash chromatography (silica gel, 0% to 50% ethyl acetate in heptane) to provide 5-cyclobutyl-N-[5-(difluoromethoxy)-4,6-dimethoxy-pyrimidin-2-yl]-1H-pyrrole-3-sulfonamide (23 mg, 48.5% yield) as white solid. MS (ESI) m/z: 405.1 [M+H]⁺.

### Example 51: 5-cyclobutyl-N-[5-(2,2-difluoroethoxy)-4,6-dimethoxy-pyrimidin-2-yl]-1H-pyrrole-3-sulfonamide

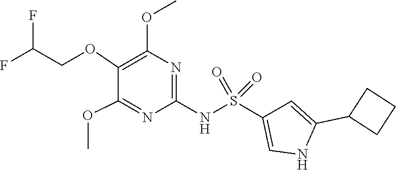 [SMILES: COc1nc(NS(=O)(=O)c2c[nH]c(C3CCC3)c2)nc(OC)c1OCC(F)F]

The title compound was prepared in analogy to Example 50 from intermediate B4 instead of intermediate B1 in step 1 as white solid, MS (ESI) m/z: 419.1 [M+H]⁺.

### REFERENCE EXAMPLES

### Reference Example RE-A

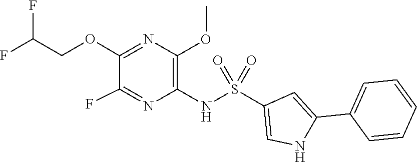 [SMILES: COc1nc(OCC(F)F)c(F)nc1NS(=O)(=O)c1c[nH]c(-c2ccccc2)c1]

RE-A is Example 436 in WO2022254027 and was synthesized following the procedure described therein.

### Reference Example RE-B

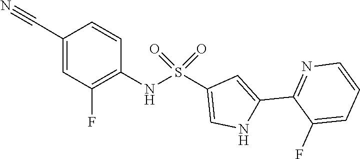 [SMILES: N#Cc1ccc(NS(=O)(=O)c2c[nH]c(-c3ncccc3F)c2)c(F)c1]

RE-B is Example 90 in WO2022254027 and was synthesized following the procedure described therein.

### Reference Example RE-C

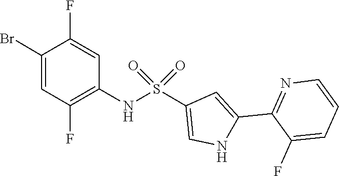 [SMILES: O=S(=O)(Nc1cc(F)c(Br)cc1F)c1c[nH]c(-c2ncccc2F)c1]

RE-C is Example 406 in WO2022254027 and was synthesized following the procedure described therein.

### Reference Example RE-D

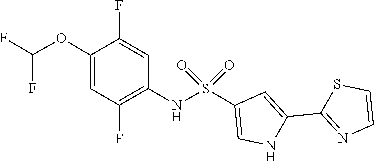 [SMILES: O=S(=O)(Nc1cc(F)c(OC(F)F)cc1F)c1c[nH]c(-c2nccs2)c1]

RE-D is Example 403 in WO2022254027 and was synthesized following the procedure described therein.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula I 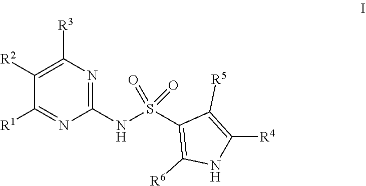
wherein,
R¹ is alkoxy or haloalkoxy;
R² is halo, alkyl, alkoxy, haloalkyl, haloalkoxy, cyanoalkyl, cyanoalkoxy, or cyclopropyl optionally substituted with up to two substituents independently selected from cyano and halo;
R³ is H, alkoxy, or haloalkoxy;
R⁴ is H, cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with oxo, halo, alkyl, haloalkyl, alkoxy, haloalkoxy, or cyano;
R⁵ is H, halo, alkyl, aryl, heteroaryl, arylalkyl or heteroarylalkyl wherein aryl, heteroaryl, arylalkyl or heteroarylalkyl are optionally substituted with halo, alkyl, haloalkyl, alkoxy, haloalkoxy, or cyano;
R⁶ is H or halo;
and pharmaceutically acceptable salts.

2. A compound according to claim 1, wherein R¹ is alkoxy.

3. A compound according to claim 1 or claim 2, wherein R² is haloalkyl, haloalkoxy, cyanoalkyl or cyclopropyl substituted with cyano.

4. A compound according to any of claims 1 to 3, wherein R² is haloalkyl or haloalkoxy.

5. A compound according to any of claims 1 to 4, wherein R³ is H or alkoxy.

6. A compound according to any of claims 1 to 5, wherein R³ is alkoxy.

7. A compound according to any of claims 1 to 6, wherein R⁴ is selected from cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with, halo, alkyl, haloalkyl, alkoxy, haloalkoxy, or cyano.

8. A compound according to any of claims 1 to 6, wherein R⁴ is selected from
i. H;
ii. Cyclobutyl;
iii. 6-membered aryl;
iv. A 5-membered heteroaryl comprising 1 S heteroatom and 1 N heteroatom;
v. A 6-membered heteroaryl comprising 1 or 2 N heteroatoms;
vi. A 6-member aryl substituted with halo;
vii. A 5-membered heteroaryl comprising 1 S heteroatom and 1 N heteroatom substituted by alkyl;
viii. A 6-membered heteroaryl comprising 1 N heteroatom substituted with alkyl, halo, or both alkyl and oxy.

9. A compound according to any of claims 1 to 6, wherein R⁴ is selected from
i. 6-membered aryl;
ii. A 5-membered heteroaryl comprising 1 S heteroatom and 1 N heteroatom;
iii. A 6-membered heteroaryl comprising 1 N heteroatoms;
iv. A 6-member aryl substituted with halo;
v. A 5-membered heteroaryl comprising 1 S heteroatom and 1 N heteroatom substituted by alkyl;
vi. A 6-membered heteroaryl comprising 1 N heteroatom substituted with alkyl or halo.

10. A compound according to any of claims 1 to 6 wherein R⁴ is a 6-membered-aryl, or a 5-or-6-membered heteroaryl optionally substituted by halo and comprising 1-to-2 heteroatoms independently selected from S and N.

11. A compound according to any of claim 10, wherein R⁵ is H, arylalkyl, or arylalkyl substituted with halo.

12. A compound according to any of claims 1 to 11 wherein R⁵ is H.

13. A compound according to any of claims 1 to 12 wherein R⁶ is H.

14. A compound according to claim 1, wherein,
R¹ is alkoxy or haloalkoxy;
R² is halo, alkyl, alkoxy, haloalkyl, haloalkoxy, cyanoalkyl, cyanoalkoxy, or cyclopropyl optionally substituted with up to two substituents independently selected from cyano and halo;
R³ is H, alkoxy, or haloalkoxy;
R⁴ is cycloalkyl, heterocycloalkyl, aryl, heteroaryl, arylalkyl or heteroarylalkyl, wherein cycloalkyl, heterocycloalkyl, aryl, heteroaryl, arylalkyl or heteroarylalkyl are optionally substituted with halo, alkyl, haloalkyl, alkoxy, haloalkoxy, or cyano;
R⁵ is H, halo, alkyl, aryl, heteroaryl, arylalkyl or heteroarylalkyl wherein aryl, heteroaryl, arylalkyl or heteroarylalkyl are optionally substituted with halo, alkyl, haloalkyl, alkoxy, haloalkoxy, or cyano;
R⁶ is H or halo;
and pharmaceutically acceptable salts.

15. A compound according to claim 1, wherein
R¹ is alkoxy;
R² is haloalkyl, haloalkoxy, cyanoalkyl or cyclopropyl substituted with cyano;
R³ is H or alkoxy;
R⁴ is
i. H;
ii. Cyclobutyl;
iii. 6-membered aryl;
iv. A 5-membered heteroaryl comprising 1 S heteroatom and 1 N heteroatom;
v. A 6-membered heteroaryl comprising 1 or 2 N heteroatoms;
vi. A 6-member aryl substituted with halo;
vii. A 5-membered heteroaryl comprising 1 S heteroatom and 1 N heteroatom substituted by alkyl;
viii. A 6-membered heteroaryl comprising 1 N heteroatom substituted with alkyl, halo, or both alkyl and oxy.
R⁵ is H, arylalkyl, or arylalkyl substituted with halo;
R⁶ is H;
and pharmaceutically acceptable salts.

16. A compound according to claim 1, wherein
R¹ is alkoxy;
R² is haloalkyl, haloalkoxy, cyanoalkyl or cyclopropyl substituted with cyano;
R³ is alkoxy;
R⁴ is
i. 6-membered aryl;
ii. A 5-membered heteroaryl comprising 1 S heteroatom and 1 N heteroatom;
iii. A 6-membered heteroaryl comprising 1 N heteroatoms;
iv. A 6-member aryl substituted with halo;
v. A 5-membered heteroaryl comprising 1 S heteroatom and 1 N heteroatom substituted by alkyl;
vi. A 6-membered heteroaryl comprising 1 N heteroatom substituted with alkyl or halo.
R⁵ is H;
R⁶ is H;
and pharmaceutically acceptable salts.

17. A compound according to claim 1, wherein
R¹ is alkoxy;
R² is haloalkyl or haloalkoxy;
R³ is alkoxy;
R⁴ is a 6-membered-aryl, or a 5-or-6-membered heteroaryl optionally substituted by halo and comprising 1-to-2 heteroatoms independently selected from S and N;
R⁵ is H;
R⁶ is H;
and pharmaceutically acceptable salts thereof.

18. A compound according to any of claims 1 to 17, selected from
N-[5-(2,2-difluoroethoxy)-4,6-dimethoxy-pyrimidin-2-yl]-5-phenyl-1H-pyrrole-3-sulfonamide;
N-[5-(difluoromethoxy)-4,6-dimethoxy-pyrimidin-2-yl]-5-phenyl-1H-pyrrole-3-sulfonamide;
N-[5-(2-fluoroethoxy)-4,6-dimethoxy-pyrimidin-2-yl]-5-phenyl-1H-pyrrole-3-sulfonamide;
N-[5-(2,2-difluoroethoxy)-4,6-dimethoxy-pyrimidin-2-yl]-5-thiazol-2-yl-1H-pyrrole-3-sulfonamide;
N-[5-(difluoromethoxy)-4,6-dimethoxy-pyrimidin-2-yl]-5-(3-fluoro-2-pyridyl)-1H-pyrrole-3-sulfonamide;
N-[5-(2,2-difluoroethyl)-4,6-dimethoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-(3-fluoro-2-pyridyl)-1H-pyrrole-3-sulfonamide;
N-[5-(2,2-difluoroethoxy)-4,6-dimethoxy-pyrimidin-2-yl]-5-(3-fluoro-2-pyridyl)-1H-pyrrole-3-sulfonamide; and
pharmaceutically acceptable salts thereof.

19. A compound according to any of claims 1 to 17, selected from
N-[5-(2,2-difluoroethoxy)-4-methoxy-pyrimidin-2-yl]-5-thiazol-2-yl-1H-pyrrole-3-sulfonamide;
N-[5-(2-cyanoethyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-isothiazol-3-yl-1H-pyrrole-3-sulfonamide;
N-[5-(2,2-difluoroethyl)-4,6-dimethoxy-pyrimidin-2-yl]-5-phenyl-1H-pyrrole-3-sulfonamide;
N-[5-(2,2-difluoroethyl)-4,6-dimethoxy-pyrimidin-2-yl]-5-thiazol-2-yl-1H-pyrrole-3-sulfonamide;
N-[5-(difluoromethoxy)-4,6-dimethoxy-pyrimidin-2-yl]-5-thiazol-2-yl-1H-pyrrole-3-sulfonamide;
N-[5-(2,2-difluoroethyl)-4,6-dimethoxy-pyrimidin-2-yl]-5-thiazol-4-yl-1H-pyrrole-3-sulfonamide;
N-[5-(difluoromethoxy)-4,6-dimethoxy-pyrimidin-2-yl]-5-thiazol-4-yl-1H-pyrrole-3-sulfonamide;
N-[5-(2,2-difluoroethyl)-4,6-dimethoxy-pyrimidin-2-yl]-5-(2-pyridyl)-1H-pyrrole-3-sulfonamide;
N-[5-(difluoromethoxy)-4,6-dimethoxy-pyrimidin-2-yl]-5-(2-pyridyl)-1H-pyrrole-3-sulfonamide;
N-[5-(2,2-difluoroethoxy)-4,6-dimethoxy-pyrimidin-2-yl]-5-(2-pyridyl)-1H-pyrrole-3-sulfonamide;
N-[5-(2,2-difluoroethyl)-4,6-dimethoxy-pyrimidin-2-yl]-5-(4-methylthiazol-2-yl)-1H-pyrrole-3-sulfonamide;
4-benzyl-N-[5-(2,2-difluoroethyl)-4,6-dimethoxy-pyrimidin-2-yl]-1H-pyrrole-3-sulfonamide;
4-benzyl-N-[5-(difluoromethoxy)-4,6-dimethoxy-pyrimidin-2-yl]-1H-pyrrole-3-sulfonamide;
N-[5-(2-cyanoethyl)-4,6-dimethoxy-pyrimidin-2-yl]-5-(3-fluoro-2-pyridyl)-1H-pyrrole-3-sulfonamide;
N-[5-(2,2-difluoroethyl)-4-methoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-thiazol-4-yl-1H-pyrrole-3-sulfonamide;
N-[5-(2,2-difluoroethoxy)-4,6-dimethoxy-pyrimidin-2-yl]-5-thiazol-4-yl-1H-pyrrole-3-sulfonamide;
N-[5-(2-fluoroethoxy)-4,6-dimethoxy-pyrimidin-2-yl]-5-(2-pyridyl)-1H-pyrrole-3-sulfonamide;
4-benzyl-N-[5-(2-fluoroethoxy)-4,6-dimethoxy-pyrimidin-2-yl]-1H-pyrrole-3-sulfonamide;
4-[dideuterio-(3-fluorophenyl)methyl]-N-[5-(2,2-difluoroethoxy)-4,6-dimethoxy-pyrimidin-2-yl]-1H-pyrrole-3-sulfonamide;
4-benzyl-N-[5-(2,2-difluoroethoxy)-4,6-dimethoxy-pyrimidin-2-yl]-1H-pyrrole-3-sulfonamide;
N-[5-(2-fluoroethoxy)-4,6-dimethoxy-pyrimidin-2-yl]-5-(4-methylthiazol-2-yl)-1H-pyrrole-3-sulfonamide;
N-[5-(difluoromethoxy)-4,6-dimethoxy-pyrimidin-2-yl]-5-(4-methylthiazol-2-yl)-1H-pyrrole-3-sulfonamide;
N-[5-(2,2-difluoroethoxy)-4,6-dimethoxy-pyrimidin-2-yl]-5-(4-methylthiazol-2-yl)-1H-pyrrole-3-sulfonamide;
N-[5-(2,2-difluoroethyl)-4-methoxy-pyrimidin-2-yl]-5-thiazol-2-yl-1H-pyrrole-3-sulfonamide;
N-[5-(2-cyanocyclopropyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-thiazol-5-yl-1H-pyrrole-3-sulfonamide;
N-[5-(2,2-difluoroethyl)-4,6-dimethoxy-pyrimidin-2-yl]-5-(2-fluorophenyl)-1H-pyrrole-3-sulfonamide;
N-[5-(2-fluoroethoxy)-4,6-dimethoxy-pyrimidin-2-yl]-5-(2-fluorophenyl)-1H-pyrrole-3-sulfonamide;
N-[5-(difluoromethoxy)-4,6-dimethoxy-pyrimidin-2-yl]-5-(2-fluorophenyl)-1H-pyrrole-3-sulfonamide;
N-[5-(2,2-difluoroethoxy)-4,6-dimethoxy-pyrimidin-2-yl]-5-(2-fluorophenyl)-1H-pyrrole-3-sulfonamide;
N-[5-(2,2-difluoroethoxy)-4,6-dimethoxy-pyrimidin-2-yl]-5-(2-keto-1-methyl-3-pyridyl)-1H-pyrrole-3-sulfonamide;
N-[5-(2,2-difluoroethoxy)-4,6-dimethoxy-pyrimidin-2-yl]-5-(2-methylthiazol-5-yl)-1H-pyrrole-3-sulfonamide;
N-[5-(2,2-difluoroethoxy)-4,6-dimethoxy-pyrimidin-2-yl]-5-pyrimidin-2-yl-1H-pyrrole-3-sulfonamide;
N-[5-(2,2-difluoroethoxy)-4,6-dimethoxy-pyrimidin-2-yl]-5-(5-methylthiazol-2-yl)-1H-pyrrole-3-sulfonamide;
N-[5-(2,2-difluoroethoxy)-4,6-dimethoxy-pyrimidin-2-yl]-5-pyridazin-3-yl-1H-pyrrole-3-sulfonamide;
5-(3-chloro-2-pyridyl)-N-[5-(2,2-difluoroethoxy)-4,6-dimethoxy-pyrimidin-2-yl]-1H-pyrrole-3-sulfonamide;
N-[5-(2,2-difluoroethoxy)-4,6-dimethoxy-pyrimidin-2-yl]-5-pyrimidin-4-yl-1H-pyrrole-3-sulfonamide;
N-[5-(2,2-difluoroethoxy)-4,6-dimethoxy-pyrimidin-2-yl]-5-pyrazin-2-yl-1H-pyrrole-3-sulfonamide;
N-[5-(2,2-difluoroethoxy)-4,6-dimethoxy-pyrimidin-2-yl]-5-(3-methyl-2-pyridyl)-1H-pyrrole-3-sulfonamide;
5-cyclobutyl-N-[5-(difluoromethoxy)-4,6-dimethoxy-pyrimidin-2-yl]-1H-pyrrole-3-sulfonamide;
5-cyclobutyl-N-[5-(2,2-difluoroethoxy)-4,6-dimethoxy-pyrimidin-2-yl]-1H-pyrrole-3-sulfonamide; and
pharmaceutically acceptable salts thereof.

20. A compound according to any of claims 1 to 19, selected from
N-[5-(2-fluoroethoxy)-4,6-dimethoxy-pyrimidin-2-yl]-5-phenyl-1H-pyrrole-3-sulfonamide;
N-[5-(2,2-difluoroethoxy)-4,6-dimethoxy-pyrimidin-2-yl]-5-thiazol-2-yl-1H-pyrrole-3-sulfonamide;
N-[5-(difluoromethoxy)-4,6-dimethoxy-pyrimidin-2-yl]-5-(3-fluoro-2-pyridyl)-1H-pyrrole-3-sulfonamide;
N-[5-(2,2-difluoroethyl)-4,6-dimethoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-(3-fluoro-2-pyridyl)-1H-pyrrole-3-sulfonamide;
N-[5-(2,2-difluoroethoxy)-4,6-dimethoxy-pyrimidin-2-yl]-5-(3-fluoro-2-pyridyl)-1H-pyrrole-3-sulfonamide;
N-[5-(2-cyanoethyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-isothiazol-3-yl-1H-pyrrole-3-sulfonamide;
N-[5-(2,2-difluoroethyl)-4,6-dimethoxy-pyrimidin-2-yl]-5-phenyl-1H-pyrrole-3-sulfonamide;
N-[5-(2,2-difluoroethyl)-4,6-dimethoxy-pyrimidin-2-yl]-5-thiazol-2-yl-1H-pyrrole-3-sulfonamide;
N-[5-(2,2-difluoroethyl)-4,6-dimethoxy-pyrimidin-2-yl]-5-(2-pyridyl)-1H-pyrrole-3-sulfonamide;
N-[5-(2,2-difluoroethoxy)-4,6-dimethoxy-pyrimidin-2-yl]-5-(2-pyridyl)-1H-pyrrole-3-sulfonamide;
N-[5-(2,2-difluoroethyl)-4,6-dimethoxy-pyrimidin-2-yl]-5-(4-methylthiazol-2-yl)-1H-pyrrole-3-sulfonamide;
N-[5-(2-cyanoethyl)-4,6-dimethoxy-pyrimidin-2-yl]-5-(3-fluoro-2-pyridyl)-1H-pyrrole-3-sulfonamide;
N-[5-(2-fluoroethoxy)-4,6-dimethoxy-pyrimidin-2-yl]-5-(2-pyridyl)-1H-pyrrole-3-sulfonamide;
N-[5-(2-fluoroethoxy)-4,6-dimethoxy-pyrimidin-2-yl]-5-(4-methylthiazol-2-yl)-1H-pyrrole-3-sulfonamide;
N-[5-(2-cyanocyclopropyl)-4,6-dimethoxy-pyrimidin-2-yl]-5-thiazol-2-yl-1H-pyrrole-3-sulfonamide;
N-[5-(2,2-difluoroethoxy)-4,6-dimethoxy-pyrimidin-2-yl]-5-thiazol-5-yl-1H-pyrrole-3-sulfonamide;
N-[5-(2,2-difluoroethyl)-4,6-dimethoxy-pyrimidin-2-yl]-5-(2-fluorophenyl)-1H-pyrrole-3-sulfonamide;
N-[5-(2-fluoroethoxy)-4,6-dimethoxy-pyrimidin-2-yl]-5-(2-fluorophenyl)-1H-pyrrole-3-sulfonamide;
5-(3-chloro-2-pyridyl)-N-[5-(2,2-difluoroethoxy)-4,6-dimethoxy-pyrimidin-2-yl]-1H-pyrrole-3-sulfonamide;
N-[5-(2,2-difluoroethoxy)-4,6-dimethoxy-pyrimidin-2-yl]-5-(3-methyl-2-pyridyl)-1H-pyrrole-3-sulfonamide; and
pharmaceutically acceptable salts thereof.

21. A process to prepare a compound according to any one of claims 1 to 20 comprising the reacting a compound of formula III with a compound of formula II in the presence of a base selected from N-ethyldiisopropylamine, pyridine, potassium phosphate or sodium hydride, to provide a compound of formula I, 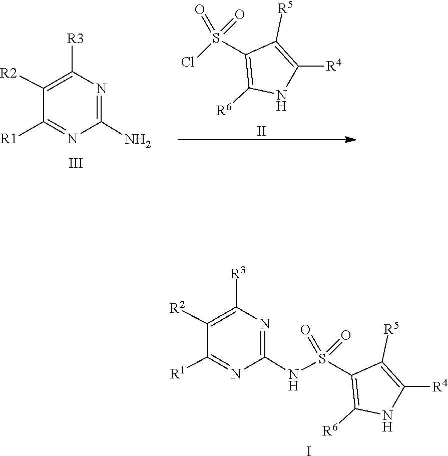
wherein R¹, R², R³, R³, R⁴, R⁵ and R⁶ are as described above.

22. A compound according to any one of claims 1 to 20 for use as therapeutically active substance.

23. A compound according to any one of claims 1 to 20 for use in the treatment of a disease modulated by GPR17.

24. A pharmaceutical composition comprising a compound according to any one of claims 1 to 20 and a therapeutically inert carrier.

25. The use of a compound according to any one of claims 1 to 20 for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity.

26. The use of a compound according to any one of claims 1 to 20 for the treatment or prophylaxis of multiple sclerosis.

27. The use of a compound according to any one of claims 1 to 20 for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity.

28. A compound according to any one of claims 1 to 20 for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity.

29. A compound according to any one of claims 1 to 20 for use in the treatment or prophylaxis of multiple sclerosis.

30. A method for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity, which method comprises administering an effective amount of a compound according to any one of claims 1 to 20 to a patient in need thereof.

31. A method for the treatment or prophylaxis of multiple sclerosis, which method comprises administering an effective amount of a compound according to any one of claims 1 to 20 to a patient in need thereof.

32. A compound according to any one of claims 1 to 20, when manufactured according to a process of claim 21.
